(19) United States
(12) Patent Application Publication
(10) Pub. No.: US 2004/0157301 A1
(43) Pub. Date: Aug. 12, 2004

(54) Methods for producing end-products from carbon substrates

(76) Inventors: Gopal K. Chotani, Cupertino, CA (US); Manoj Kumar, Fremont, CA (US); Jeff P. Pucci, Pacifica, CA (US); Karl J. Sanford, Cupertino, CA (US); Jayarama K. Shetty, Pleasanton, CA (US)

Correspondence Address: KAMRIM T. MACKNIGHT, GENENCOR INTERNATIONAL, INC., 925 PAGE MILL ROAD, PALO ALTO, CA, 94304-1013, US

(21) Appl. No.: 10/765,652

(22) Filed: Jan. 26, 2004

Related U.S. Application Data
(63) Continuation of application No. 10/359,771, filed on Feb. 6, 2003.
(60) Provisional application No. 60/355,260, filed on Feb. 8, 2002.
(60) Provisional application No. 60/355,180, filed on Feb. 8, 2002.

(51) Int. Cl. C12P 19/02; C12P 7/06; C12N 1/21

(52) U.S. Cl. 435/105; 435/161; 435/252.3

(57) ABSTRACT

The present invention provides means for the production of desired end-products of in vitro and/or in vivo bioconversion of biomass-based feed stock substrates, including but not limited to such materials as starch and cellulose. In particularly preferred embodiments, the methods of the present invention do not require gelatinization and/or liquefaction of the substrate.

The present application claims priority to U.S. Prov. Patent Appln. Ser. No. 60/355,260, filed Feb. 8, 2002, as well as U.S. Prov. Patent Appln. Ser. No. 60/355,180, filed Feb. 8, 2002.

### FIELD OF THE INVENTION

The present invention provides means for the production of desired end-products of in vitro and/or in vivo bioconversion of biomass-based feed stock substrates, including but not limited to such materials as starch and cellulose. In particularly preferred embodiments, the methods of the present invention do not require gelatinization and/or liquefaction of the substrate.

### BACKGROUND OF THE INVENTION

Industrial fermentations predominantly use glucose as feed-stock for the production of proteins, enzymes and chemicals. These fermentations are usually batch, fed-batch, or continuous, and operate under substrate-limited and minimal by-products forming conditions. These are critical operating conditions that must be controlled during fermentation in order to optimize fermentation time, yield and efficiency. Currently used methods and feed-stocks have drawbacks that reduce the efficiency of the fermentation processes.

Glucose is a natural, carbon based compound that is useful in a multitude of chemical and biological synthetic applications as a starting substrate. However, syrups that contain glucose purity levels of greater than 90% are relatively expensive. In addition, the presence of high glucose concentrations increases the susceptibility of the fermentation system to microbial contamination, thereby resulting in an adverse effect upon the production efficiency. Another disadvantage is that even the presence of low to moderate levels of glucose in the fermentation vat adversely affects the conversion of the glucose to the desired end product, for example by enzymatic inhibition and/or catabolite repression, and/or the growth of microorganisms. As a result, various attempts have been made to reduce the costs of industrial fermentation, particularly in utilization of less expensive substrates than glucose. However, despite the development of numerous approaches, there remains a need in the art for economical, efficiently-utilized substrates for fermentation. Indeed, there is a great need in the art for methods that utilize a less expensive starting material than glucose to more efficiently produce a desired end-product.

### SUMMARY OF THE INVENTION

The present invention provides means for the production of desired end-products of in vitro and/or in vivo bioconversion of biomass-based feed stock substrates, including but not limited to such materials as starch and cellulose. In particularly preferred embodiments, the methods of the present invention do not require gelatinization and/or liquefaction of the substrate.

In some preferred embodiments, the present invention provides methods for producing an end-product characterized by maintaining the intermediate concentration of the conversion at a low concentration, preferably below the threshold triggering catabolite repression and/or enzyme inhibition, so as to increase efficiency of the process by avoiding catabolic repressive and/or enzymatic inhibitive effects of the intermediate upon the enzymatic conversion of the substrate to the end-product.

In some particularly preferred embodiments, the present invention provides methods for producing an end-product, including organic acids, including but not limited to gluconic acid, ascorbic acid intermediates, succinic acid, citric acid, acetic acid, lactic acid, amino acids, and antimicrobials, as well as enzymes and organic solvents, including but not limited to 1,3-propanediol, butanol, acetone, glycerol, and ethanol. In some embodiments, the methods comprise the steps of contacting a carbon substrate and at least one substrate converting enzyme to produce an intermediate; and then contacting the intermediate with at least one intermediate producing enzyme, wherein the intermediate is substantially completely bioconverted by an end-product producing microorganism. In additional embodiments, the substrate-converting and/or intermediate-converting enzyme(s) are provided as a cell-free extract.

In some preferred embodiments, production of end-products is efficiently accomplished by maintaining a low concentration of the intermediate in a conversion medium, such that catabolite repression and/or enzyme inhibition effects associated with intermediate product formation are reduced. The present invention provides methods in various levels of intermediate concentration, substrates, intermediates and steps of converting the intermediate to ethanol are provided.

The present invention provides methods for producing end-products comprising the steps of: contacting a carbon substrate and at least one substrate-converting enzyme to produce an intermediate; and contacting the intermediate with at least one intermediate-converting enzyme, wherein the intermediate is substantially all converted by the intermediate enzyme to an end-product. In some preferred embodiments, the intermediate-converting enzyme is a microbial enzyme. In some alternative embodiments, the microbial enzyme is produced in by a microorganism in contact with the intermediate. In some additional embodiments, the substrate-converting enzyme is a microbial enzyme. In further embodiments, the microbial enzyme is produced by a microorganism in contact with the substrate. In still further embodiments, both the substrate-converting enzyme and the intermediate-converting enzyme are produced by a microorganism in contact with the intermediate and/or the substrate. In some embodiments, both enzymes are provided by the same species of microorganism, while in other embodiments, the enzymes are produced by microorganisms of different species. In some particularly preferred embodiments, the concentration level of the intermediate is maintained at a level below that which triggers catabolite repression effects upon the conversion of the intermediate to the end-product. In further preferred embodiments, the concentration level of the intermediate is maintained at a level below that which triggers enzymatic inhibition effects upon the conversion of the intermediate to the end-product. In still other embodiments, the intermediate is converted to the end-product at a rate sufficient to maintain the concentration of at less than 0.25% of the mixture. In some particularly preferred embodiments, the substrate is selected from the group consisting of biomass and starch. In still further embodiments, the intermediate is selected from the group consisting of hexoses and pentoses. In some preferred embodiments, the hexose is glucose. In various preferred embodiments, the end-product is selected from the group consisting of 1,3-propanediol, gluconic acid, glycerol, succinic acid, lactic acid, 2,5-diketo-D-gluconic acid, gluconate, glucose, alcohol, and ascorbic acid intermediates. In other embodiments, more than one end-product is produced. In still further embodiments, the step of contacting the substrate and at least one substrate-converting enzyme further comprises bioconverting the substrate to produce the intermediate. In some embodiments, more than one intermediate is produced. In this case, in some embodiments, the intermediate-converting enzyme(s) work on all of the intermediates, while in other embodiments, the intermediate-converting enzyme(s) work on a subset of the intermediates, while in further embodiments, the intermediate-converting enzyme(s) work on only one of the intermediates to produce at least one end-product. In additional embodiments, the substrate-converting and/or intermediate-converting enzyme(s) are provided as a cell-free extract.

The present invention also provides methods for producing an end-product comprising the steps of contacting a carbon substrate and at least one substrate-converting enzyme to produce an intermediate; and contacting the intermediate with at least one intermediate-converting enzyme, wherein the intermediate is substantially all converted by the intermediate enzyme to an end-product, and wherein the presence of the end-product does not inhibit the further production of the end-product. In some embodiments, more than one intermediate is produced. In this case, in some embodiments, the intermediate-converting enzyme(s) work on all of the intermediates, while in other embodiments, the intermediate-converting enzyme(s) work on a subset of the intermediates, while in further embodiments, the intermediate-converting enzyme(s) work on only one of the intermediates to produce at least one end-product. In some embodiments, the intermediate-converting enzyme is a microbial enzyme, while in other embodiments the substrate-converting enzyme is a microbial enzyme. In some preferred embodiments, the substrate-converting and/or intermediate converting enzymes are produced by a microorganism in contact with the intermediate and/or the substrate. In some embodiments, both enzymes are provided by the same species of microorganism, while in other embodiments, the enzymes are produced by microorganisms of different species. In additional embodiments, the substrate-converting and/or intermediate-converting enzyme(s) are provided as a cell-free extract.

The present invention also provides methods for producing an end-product comprising the steps of: contacting a carbon substrate and at least one substrate-converting enzyme to produce an intermediate; and contacting the intermediate with at least one intermediate-converting enzyme, wherein the intermediate is substantially all converted by the intermediate enzyme to an end-product, and wherein the presence of the substrate does not inhibit the further production of the end-product. In some embodiments, the intermediate-converting enzyme is a microbial enzyme, while in other embodiments the substrate-converting enzyme is a microbial enzyme. In some preferred embodiments, the substrate-converting and/or intermediate converting enzymes are produced by a microorganism in contact with the intermediate and/or the substrate. In some embodiments, both enzymes are provided by the same species of microorganism, while in other embodiments, the enzymes are produced by microorganisms of different species. In some embodiments, more than one intermediate is produced. In this case, in some embodiments, the intermediate-converting enzyme(s) work on all of the intermediates, while in other embodiments, the intermediate-converting enzyme(s) work on a subset of the intermediates, while in further embodiments, the intermediate-converting enzyme(s) work on only one of the intermediates to produce at least one end-product. In additional embodiments, the substrate-converting and/or intermediate-converting enzyme(s) are provided as a cell-free extract.

In some preferred embodiments, the contacting steps take place in a reaction vessel, including but not limited to vats, bottles, flasks, bags, bioreactors, and any other receptacle suitable for conducting the methods of the present invention.

### DESCRIPTION OF THE FIGURES

FIG. 1 provides a graph showing the bioconversion of glucose to gluconic acid by the enzymes OXYGO® and FERMCOLASE® in a batch bioreactor.

FIG. 2 provides a graph showing the bioconversion of raw corn starch to D-glucose by CU CONC RSH glucoamylase (Shin Nihon Chemicals, Japan) in a batch bioreactor.

FIG. 3 provides a graph showing the bioconversion of raw corn starch to D-gluconate in the presence of CU CONC, OXYGO®, and FERMCOLASE® enzymes in a batch bioreactor.

FIG. 4 provides a graph showing the bioconversion of starch to gluconic acid in the presence of CU CONC, OXYGO®, FERMCOLASE®, and DISTILLASE® enzymes under modified conditions in a batch bioreactor.

FIG. 5 provides a graph showing the bioconversion of maltodextrin to glucose by OPTIMAX® 4060 in a batch bioreactor.

FIG. 6 provides a graph showing results from an enzyme dosage analysis to determine the appropriate enzyme concentration for the most efficient bioconversion of glucose to gluconate.

FIG. 7 provides a graph showing the bioconversion of maltodextrin to gluconate under modified enzyme dosages.

FIG. 8 provides a graph showing the optimization of enzyme dosage to improve overall conversion of maltodextrin to gluconate.

FIG. 9 provides a graph showing the bioconversion of starch to 2,5-diketo gluconic acid (DKG).

FIG. 10, provides graph showing the bioconversion of granular starch to glucose and lactate.

FIG. 11 provides a graph showing the biocatalytic conversion of granular starch to glucose and its conversion to succinate.

FIG. 12 provides a graph showing the bioconversion of granular starch to glucose, its conversion to glycerol, and then to 1,3-propanediol.

FIG. 13 provides a graph showing the bioconversion of granular starch to glucose formation, its conversion to glycerol, and then to 1,3-propanediol.

FIG. 14 provides a graph showing bioconversion of granular starch to glycerol.

FIG. 15 provides a graph showing bioconversion of corn starch to glucose and its conversion to 2,5-diketo-D-gluconic acid.

FIG. 16(A), provides a graph showing the biconversion of cellulose (AVICEL®) to glucose by SPEZYME® enzyme.

FIG. 16(B) provides a graph showing the biocatalytic conversion of cellulose (AVICEL®) to gluconic acid by SPEZYME™ (“SPE”), OXYGO® and FERMCOLASE® enzymes.

FIG. 16(C) provides a graph showing the biocatalytic conversion of corn stover to gluconic acid by SPEZYME® (“SPE”), OXYGO® and FERMCOLASE® enzymes.

FIG. 16(D) provides a graph showing the biocatalytic conversion of cellulose (AVICEL®) to gluconic acid by SPEZYME® (“SPE”), OXYGO® and FERMCOLASE® enzymes.

FIG. 17 provides a graph showing the bioconversion of cellulose to glycerol and 1,3-propanediol.

FIG. 18 provides a graph showing the bioconversion of cellulose to lactate.

FIG. 19 provides a graph showing the bioconversion of cellulose to succinate.

### BRIEF DESCRIPTION OF THE INVENTION

The present invention provides means for the production of desired end-products of in vitro and/or in vivo bioconversion of biomass-based feed stock substrates, including but not limited to such materials as starch and cellulose. In particularly preferred embodiments, the methods of the present invention do not require gelatinization and/or liquefaction of the substrate.

The present invention provides methods in which starches or biomass and hydrolyzing enzymes are used to convert starch or cellulose to glucose. In addition, the present invention provides methods in which these substrates are provided at such a rate that the conversion of starch to glucose matches the glucose feed rate required for the respective fermentative product formation. Thus, the present invention provides key glucose-limited fermentative conditions, as well as avoiding many of the metabolic regulations and inhibitions.

In some preferred embodiments, the present invention provides means for making desired end-products, in which a continuous supply of glucose is provided under controlled rate conditions, providing such benefits as reduced raw material cost, lower viscosity, improved oxygen transfer for metabolic efficiency, improved bioconversion efficiency, higher yields, altered levels of catabolite repression and enzymatic inhibition, and lowered overall manufacturing costs.

As indicated above, there is a great need in the art for methods in which less expensive starting materials than glucose are used to efficiently produce a desired end-product. As described in greater detail herein, the present invention provides methods involving such substrates, including starch (e.g., corn and wheat starch) and biomass.

Starch is a plant-based fermentation carbon source. Corn starch and wheat starch are carbon sources that are much cheaper than glucose carbon feedstock for fermentation. Conversion of liquefied starch to glucose is known in the art and is generally carried out using enzymes such alpha-amylase, pullulanase, and glucoamylase. A large number of processes have been described for converting liquefied starch to the monosaccharide, glucose. Glucose has value in itself, and also as a precursor for other saccharides such as fructose. In addition, glucose may also be fermented to ethanol or other fermentation products. However the ability of the enzymatic conversion of a first carbon source to the intermediate, especially glucose, may be impaired by the presence of the intermediate.

For example, the typical methods used in Japanese sake brewing and alcoholic production use starch without cooking. However, these techniques require some special operations such as acidification of mash (pH 3.5), which prevents contamination of harmful microorganisms. Furthermore, these methods require a longer period of the time for the saccharification and fermentation than the present invention. In addition, these methods require complex process steps such as dialysis of a fermented broth and are too cumbersome to utilize in the general production of products via fermentation.

The use of soluble dextrins and glucose as feed-stock in fermentations have various drawbacks, including high processing cost, and problems associated with viscosity and oxygen transfer. In addition, in comparison to the present invention, these methods produce lower yields of the desired products and more problems associated with the formation of by-products. Indeed, the costs of converting starch or biomass to dextrins are substantial and involve high energy input, separate reactor tanks, more time, a detailed bioprocess operation, incomplete saccharification, back-reaction, and enzymes associated with the typical pre-fermentation saccharification step. These problems have led to a number of attempts to provide methods for conversion directly to starch within one reaction vessel or container and at lower temperatures. Biotransformation of a carbohydrate source to 1,3-propanediol in mixed cultures is described in U.S. Pat. No. 5,599,689 (Haynie, et al.). The method described by Haynie et al., involves mixing a glycerol (i.e., an intermediate) producing organism with a diol producing organism (i.e., an end-product), contacting the mixed culture medium with a carbon substrate and incubating the mixed culture medium to produce the desired end-product, 1,3-propanediol. In U.S. Pat. No. 4,514,496, Yoshizuma describes methods that involve maintaining the concentration of the raw material in the slurry relative the mashing liquid to produce alcohol by fermentation without cooking (i.e., without high temperature liquefaction before saccharization. Nonetheless, these methods lack the efficiency and economical advantages provided by the present invention.

The present invention provides methods for producing end-products, including organic acids (e.g., gluconic acid, ascorbic acid intermediates, succinic acid, citric acid, acetic acid, gluconic acid, and lactic acid), amino acids, antibiotics, enzymes and organic solvents (e.g., 1,3-propanediol, butanol, and acetone), glycerol, and ethanol are provided. In some preferred embodiments, the methods comprise the steps of contacting at least one carbon substrate with at least one substrate converting enzyme to produce at least one intermediate; and contacting the at least one intermediate with an intermediate producing enzyme (typically within a reaction vessel of any suitable type), wherein the at least one intermediate is substantially completely bioconverted an end-product. In some preferred embodiments, this bioconversion is achieved by microorganisms. By maintaining a low concentration of the intermediate in a conversion medium, the intermediate's catabolite repressive and/or enzymatic inhibitive effects are altered (e.g., reduced). The present invention also provides various levels of intermediate concentration, substrates, intermediates and steps of converting the intermediate to the desired end-product.

Definitions

Unless defined otherwise herein, all technical and scientific terms used herein have the same meaning as commonly understood by one of ordinary skill in the art to which this invention belongs. Various references (See e.g., Singleton, et al., DICTIONARY OF MICROBIOLOGY AND MOLECULAR BIOLOGY, 2D ED., John Wiley and Sons, New York [1994]; and Hale and Marham, THE HARPER COLLINS DICTIONARY OF BIOLOGY, Harper Perennial, NY [1991]) provide general definitions of many of the terms used herein. Furthermore, all patents and publications, including all sequences disclosed within such patents and publications, referred to herein are expressly incorporated by reference.

Although any methods and materials similar or equivalent to those described herein find use-in the practice of the present invention, preferred methods and materials are described herein. Numeric ranges are inclusive of the numbers defining the range. Unless otherwise indicated, nucleic acids are written left to right in 5′ to 3′ orientaton; amino acid sequences are written left to right in amino to carboxy orientation, respectively. It is to be understood that this invention is not limited to the particular methodology, protocols, and reagents described, as these may vary.

The headings provided herein are not limitations of the various aspects or embodiments of the invention which can be had by reference to the specification as a whole. Furthermore, the terms defined immediately below are more fully defined by reference to the Specification as a whole.

As used herein, the term “carbon substrate” refers to a material containing at least one carbon atom which can be enzymatically converted into an intermediate for subsequent conversion into the desired carbon end-product. Exemplary carbon substrates include, but are not limited to biomass, starches, dextrins and sugars.

As used herein, “biomass” refers to cellulose- and/or starch-containing raw materials, including but not limited to wood chips, corn stover, rice, grasses, forages, perrie-grass, potatoes, tubers, roots, whole ground corn, cobs, grains, wheat, barley, rye, milo, brans, cereals, sugar-containing raw materials (e.g., molasses, fruit materials, sugar cane or sugar beets), wood, and plant residues. Indeed, it is not intended that the present invention be limited to any particular material used as biomass. In preferred embodiments of the present invention, the raw materials are starch-containing raw materials (e.g., cobs, whole ground corns, corns, grains, milo, and/or cereals, and mixtures thereof). In particularly preferred embodiments, the term refers to any starch-containing material originally obtained from any plant source.

As used herein, “starch” refers to any starch-containing materials. In particular, the term refers to various plant-based materials, including but not limited to wheat, barley, potato, sweet potato, tapioca, corn, maize, cassava, milo, rye, and brans. Indeed, it is not intended that the present invention be limited to any particular type and/or source of starch. In general, the term refers to any material comprised of the complex polysaccharide carbohydrates of plants, comprised of amylose and amylopectin, with the formula (C₆H₁₀O₅)ₓ, wherein “x” can be any number.

As used herein, “cellulose” refers to any cellulose-containing materials. In particular, the term refers to the polymer of glucose (or “cellobiose”), with the formula (C₆H₁₀O₅), wherein “x” can be any number. Cellulose is the chief constituent of plant cell walls and Is among the most abundant organic substances in nature. While there is a β-glucoside linkage in cellulose, there is a an a-glucoside linkage in starch. In combination with lignin, cellulose forms “lignocellulose.”

As used herein, “intermediate” refers to a compound that contains at least one carbon atom into which the carbon substrates are enzymatically converted. Exemplary intermediates include, but are not limited to pentoses (e.g., xylose, arabinose, lyxose, ribose, ribulose, xylulose); hexoses (e.g., glucose, allose, altrose, mannose, gulose, idose, galactose, talose, psicose, fructose, sorbose, and tagatose); and organic acids thereof.

As used herein, the term “enzymatic conversion” refers to the modification of a carbon substrate to an intermediate or the modification of an intermediate to an end-product by contacting the substrate or intermediate with an enzyme. In some embodiments, contact is made by directly exposing the substrate or intermediate to the appropriate enzyme. In other embodiments, contacting comprises exposing the substrate or intermediate to an organism that expresses and/or excretes the enzyme, and/or metabolizes the desired substrate and/or intermediate to the desired intermediate and/or end-product, respectively.

As used herein, the term “starch hydrolyzing enzyme” refers to any enzyme that is capable of converting starch to the intermediate sugar (e.g., a hexose or pentose).

As used herein, “monosaccharide” refers to any compound having an empirical formula of (CH₂O)ₙ, wherein n is 3-7, and preferably 5-7. In some embodiments, the term refers to “simple sugars” that consist of a single polyhydroxy aldehyde or ketone unit. The term encompasses, but is not limited to such compounds as glucose, galactose, and fructose.

As used herein, “disaccharide” refers to any compound that comprises two covalently linked monosaccharide units. The term encompasses, but is not limited to such compounds as sucrose, lactose and maltose.

As used herein, “oligosaccharide” refers to any compound having 2-10 monosaccharide units joined in glycosidic linkages. In some preferred embodiments, the term refers to short chains of monosaccharide units joined together by covalent bonds.

As used herein, “polysaccharide” refers to any compound having multiple monosaccharide units joined in a linear or branched chain. In some preferred embodiments, the term refers to long chains with hundreds or thousands of monosaccharide units. Some polysaccharides, such as cellulose have linear chains, while others (e.g., glycogen) have branched chains. Among the most abundant polysaccharides are starch and cellulose, which consist of recurring glucose units (although these compounds differ in how the glucose units are linked).

As used herein, “culturing” refers to fermentative bioconversion of a carbon substrate to the desired end-product (typically within a reaction vessel). In particularly preferred embodiments, culturing involves the growth of microorganisms under suitable conditions for the production of the desired end-product(s).

As used herein, the term “saccharification” refers to converting a directly unusable polysaccharide to a useful sugar feed-stock for bioconversion or fermentative bioconversion.

As used herein, the term “fermentation” refers to the enzymatic and anaerobic breakdown of organic substances by microorganisms to produce simpler organic products. In preferred embodiments, fermentation refers to the utilization of carbohydrates by microorganisms (e.g., bacteria) involving an oxidation-reduction metabolic process that takes place under anaerobic conditions and in which an organic substrate serves as the final hydrogen acceptor (i.e., rather than oxygen). Although fermentation occurs under anaerobic conditions, it is not intended that the term be solely limited to strict anaerobic conditions, as fermentation also occurs in the presence of oxygen.

As used herein, the terms “substantially all consumed” and “substantially all bioconverted” refer to the maintenance of a low level of intermediate in a conversion medium which adversely affects the enzymatic inhibition, oxygen transfer, yield, by-product minimization and/or catabolite repression effects of the intermediate (e.g., a hexose), upon the ability of the intermediate converting enzyme to convert the intermediate to the end-product or another intermediate and/or the ability of the substrate converting enzyme to convert the substrate to the intermediate.

As used herein, the terms “bioconversion” and “bioconverted” refer to contacting a microorganism with the carbon substrate or intermediate, under conditions such that the carbon substrate or intermediate is converted to the intermediate or desired end-product, respectively. In some embodiments, these terms are used to describe the production of another intervening intermediate in in vitro methods in which biocatalysts alone are used. In some preferred embodiments, the terms encompass metabolism by microorganisms and/or expression or secretion of enzyme(s) that achieve the desired conversion.

As used herein, the terms “conversion media” and “conversion medium” refer to the medium/media in which the enzymes and the carbon substrate, intermediate and end-products are in contact with one another. These terms include, but are not limited to fermentation media, organic and/or aqueous media dissolving or otherwise suspending the enzymes and the carbon substrate, intermediate and end-products. In some embodiments, the media are complex, while in other preferred embodiments, the media are defined.

As used herein, the term “end-product” refers to any carbon-source derived. molecule product which is enzymatically converted from the intermediate. In particularly preferred embodiments, the methods of the present invention are used in order to produce a “desired end-product” (i.e., the product that is intended to be produced through the use of these methods).

As used herein, “low concentration” refers to a concentration level of a compound that is less than that would result in the production of detrimental effects due to the presence of the compound. In particularly preferred embodiments, the term is used in reference to the concentration of a particular intermediate below which the detrimental effects of catabolite suppression and/or enzyme inhibition are observed. In some embodiments, the term refers to the concentration level of a particular intermediate above which triggers catabolite repression and/or enzymes inhibition by substrate and/or products.

As used herein, the phrase “maintained at a level below which triggers catabolite repression effects” refers to maintaining the concentration of an intermediate to below that level which triggers catabolite repression.

As used herein, the term “reduces catabolite repression” means conditions under which the effects of catabolite repression are produced. In preferred embodiments, the term refers to conditions in which the intermediate concentration is less than that threshold which triggers catabolite repressive effects.

As used herein, the term “reduces enzyme inhibition” means conditions under which the inhibition of an enzyme is reduced as compared to the inhibition of the enzyme under usual, standard conditions. In preferred embodiments of the present invention, the term refers to conditions in which the concentration of an intermediate, substrate and/or product of the enzyme reaction is less than that threshold which triggers enzyme inhibition.

As used herein, the term “substrate converting enzyme” refers to any enzyme that converts the substrate (e.g., granular starch) to an intermediate, (e.g., glucose). Substrate converting enzymes include, but are not limited to alpha-amylases, glucoamylases, pullulanases, starch hydrolyzing enzymes and various combinations thereof.

As used herein, the term “intermediate converting enzyme” refers to any enzyme that converts an intermediate (e.g., D-glucose, D-fructose, etc.), to the desired end-product. In preferred embodiments, this conversion is accomplished through hydrolysis, while in other embodiments, the conversion involves the metabolism of the intermediate to the end-product by a microorganism. However, it is not intended that the present invention be limited to any particular enzyme or means of conversion. Indeed, it is intended that any appropriate enzyme will find use in the various embodiments of the present invention.

As used herein, “yield” refers to the amount of end-product or intermediate produced using the methods of the present invention. In some preferred embodiments, the yield produced using the methods of the present invention is greater than that produced using methods known in the art. In some embodiments, the yield refers to the volume of the end-product or intermediate, while in other embodiments, the term is used in reference to the concentration of the end-product or intermediate in a composition.

As used herein, the term “oxygen transfer” refers to having sufficient dissolved oxygen in the bioconversion and/or fermentative bioconversion medium transferred form gas phase to a liquid medium such that it is not a rate limiting step.

As used herein, “by-product formation” refers to the production of products that are not desired. In some preferred embodiments, the present invention provides methods that avoid or reduce the production of by-products, as compared to methods known in the art.

As used herein, the term “enzymatic inhibition” refers to loss of enzyme activity by either physical or biochemical effects on the enzyme. In some embodiments, inhibition results from the effects of the product formed by the enzyme activity, while in other embodiments, inhibition results from the action of the substrate or intermediate on the enzyme.

As used herein, “enzyme activity” refers to the action of an enzyme on its substrate. In some embodiments, the enzyme activity is quantitated using means to determine the conversion of the substrate to the intermediate, while in other embodiments, the conversion of the substrate to the end-product is determined, while in still further embodiments, the conversion of the intermediate to the end-product is determined.

As used herein, the term “enzyme unit” refers to the amount of enzyme which converts 1 micromole of substrate per minute to the substrate product at optimum assay conditions (unless otherwise noted). In some embodiments, commercially available enzymes (e.g., SPEZYME®, DISTALLASE®, OPTIMAX®; Genencor International) find use in the methods of the present invention.

As used herein, the term “glucoamylase unit” (GAU) is defined as the amount of enzyme required to produce one micromole of glucose per minute under assay conditions of 40° C. and pH 5.0.

As used herein, the term “glucose oxidase unit” (GOU) is defined as the amount of enzyme required to oxidize one micromole of D-glucose per minute under assay conditions of 25° C. and pH 7.0, to gluconic acid.

As used herein, the term “catalase units” (CU) is defined as the amount of enzyme required to decompose 1 micromole of hydrogen peroxide per minute under assay conditions of 25° C. and pH 7.0.

As used herein, one AG unit (GAU) is the amount of enzyme which splits one micromole of maltose per minute at 25° C. and pH 4.3. In some embodiments of the present invention, a commercially available liquid form of glucoamylase (OPTIDEX® L-400; Genencor International) with an activity of 400 GAU per ml is used.

As used herein, “carbon end-product” means any carbon product produced from the carbon intermediate, wherein the substrate contains at least one carbon atom (i.e., a carbon substrate).

As used herein, “carbon intermediate” refers to the carbon-containing compounds that are produced during the conversion of a carbon-containing substrate to a carbon end-product.

As used herein, “enzymatically controlled” means regulating the amount of carbon intermediate produced from the carbon substrate by altering the amount or activity of the enzyme used in the reaction.

As used herein, “microorganism” refers to any organism with cells that are typically considered to be microscopic, including such organisms as bacteria, fungi (yeasts and molds), rickettsia, and protozoa. It is not intended that the present invention be limited to any particular microorganism(s) or species of microorganism(s), as various microorganisms and microbial enzymes are suitable for use in the present invention. It is also not intended that the present invention be limited to wild-type microorganisms, as microorganisms and microbial enzymes produced using recombinant DNA technologies also find use in the present invention.

As used herein, “microbial enzyme” refers to any enzyme that is produced by a microorganism. As used herein, a “microbial intermediate-converting enzyme” is an enzyme that converts an intermediate to an end-product, while a “microbial substrate-converting enzyme” is an enzyme that converts a substrate to an intermediate or directly converts a substrate to an end-product (i.e., there is not intermediate compound).

As used herein, “gluconic acid” refers to an oxidative product of glucose, wherein the C6 hydrozyl group of glucose is oxidized to a carboxylic acid group.

As used herein, the terms “gluconic acid producer” and “gluconic acid producing organism” refers to any organism or cell that is capable of producing gluconic acid through the use of a hexose or a pentose. In some embodiments, gluconic acid producing cells contain a cellulase as a substrate converting enzyme, and glucose oxidase and catalase for the conversion of the intermediates to gluconic acid.

As used herein, “glycerol producer” and “glycerol producing organism” refer to any organism or cell capable of producing glycerol. In some embodiments, glycerol producing organisms are aerobic bacteria, while in other embodiments, they are anaerobic bacteria. In still further embodiments, glycerol producing organisms include microorganisms such as fungi (i.e., molds and yeast), algae and other suitable organisms.

As used herein, the terms “diol producer,” “propanediol producer,” “diol producing organism,” and “propanediol producing organism” refer to any organism that is capable of producing 1,3-propanediol utilizing glycerol. Generally, diol producing cells contain either a diol dehydratase enzyme or a glycerol dehydratase enzyme.

As used herein, the terms “lactate producer,” and “lactate producing organism,” and “lactate producing microorganism” refer to any organism or cell that is capable of producing lactate by utilizing a hexose or a pentose. In some embodiments, the lactate producers are members of the genera Lactobacillus or Zymomonas, while in other embodiments, they organisms are fungi.

As used herein, the terms “ethanol producer” and “ethanol producing organism” refer to any organism or cell that is capable of producing ethanol from a hexose or a pentose. Generally, ethanol producing cells contain an alcohol dehydrogenase and pyruvate decarboxylase.

As used herein, the term “ascorbic acid intermediate producer” and “ascorbic acid intermediate producing organism” refers to any organism or cell that is capable of producing an ascorbic acid intermediate from a hexose or a pentose. Generally, ethanol producing cells contain a glucose dehydrogenase, gluconic acid dehydrogenase, 2,5-diketo-D-gluconate reductase, 2-keto-D-gluconate reductase, 2-keto-reductase, 5-keto reductase, glucokinase, glucono kinase, ribulose-5-phosphate epimerase, transketolase, transaldolase, hexokinase, 2,5-DKG reductase, and/or idonate dehydrogenase, depending upon the specific ascorbic acid intermediate desired.

As used herein, the term “ascorbic acid intermediate intermediate” refers to any of the following compounds: D-gluconate, 2-keto-D-gluconate (2KDG), 2,5-diketo-D-gluconate (2,5-DKG or5 DKG), 2-keto-L-gulonic acid (2KLG or KLG), L-idonic acid (IA), erythorbic acid (EA), and ascorbic acid (ASA).

As used herein, “citric acid” refers to having the formula C₆H₈O₇, commonly found in citrus fruits, beets, cranberries and other acid fruits. The term refers to citric acid from any source, whether natural or synthetic, as well as salts and any other form of the acids.

As used herein, “succinic acid” refers to the acid having the formula C₄H₆O₄, which is commonly found in amber, algae, lichens, sugar cane, beets and other plants. This acid is also formed during the fermentation of sugar, tartrates, malates, and other substances by various molds, yeasts and bacteria. The term refers to succinic acid from any source, whether natural or synthetic, as well as acid and neutral salts and esters, and any other form of the acid.

As used herein, “amino acid” refers to any of naturally-occurring amino acids, as well as any synthetic amino acids, including amino acid derivatives.

As used herein, “antimicrobial” refers to any compound that kills or inhibits the growth of microorganisms (including but not limited to antibacterial compounds).

As used herein, the term “linked culture” refers to a fermentation system that employs at least two cell cultures, in which the cultures are added sequentially. In most embodiments of linked systems, a primary culture or a set of primary cultures is grown under optimal fermentation conditions for the production of a desired intermediate (i.e., the intermediate is released into the culture media to produce a “conditioned medium”). Following the fermentation of the primary culture, the conditioned medium is then exposed to the secondary culture(s). The secondary cultures then convert the intermediate in the conditioned media to the desired end-product. In some embodiments of the present invention, the primary cultures are typically glycerol producers and the secondary cultures are 1,3-propanediol producers.

As used herein, “mixed culture” refers to the presence of any combination of microbial species in a culture. In some preferred embodiments, the mixed culture is grown in a reaction vessel under conditions such that the interaction of the individual metabolic processes of the combined organisms results in a product which neither individual organism is capable of producing. It is not intended that the present invention be limited to mixed cultures comprising a particular number of microbial species.

As used herein, “conditioned media” refers to any fermentation media suitable for the growth of microorganisms that has been supplemented by organic by-products of microbial growth. In preferred embodiments of the present invention, conditioned media are produced during fermentation of linked cultures wherein glycerol producing cells secrete glycerol into the fermentation media for subsequent conversion to 1,3-propanediol.

As used herein, “oxygen uptake rate” (“OUR”) refers to the determination of the specific consumption of oxygen within a reaction vessel. Oxygen consumption can be determined using various on-line measurements known in the art. In one embodiment, the OUR (mmol/(liter*hour)) is determined by the following formula: ((Airflow (standing liters per minute)/Fermentation weight (weight of the fermentation broth in kilograms))×supply O₂×broth density×(a constant to correct for airflow calibration at 21.1 C instead of standard 20.0 C)) minus ([airflow /fermentation weight]×[offgas O₂/offgas N₂]×supply N₂×broth density×constant).

As used herein, “carbon evolution rate” (“CER”) refers to the determination of how much CO₂ is produced within a reaction vessel during fermentation. Usually, since no CO₂ is initially or subsequently provided to the reaction vessel, any CO₂ is assumed to be produced by the fermentation process occurring within the reaction vessel. “Off-gas CO₂” refers to the amount of CO₂ measured within a reaction vessel, usually by mass spectroscopic methods known in the art.

As used herein, the term “enhanced” refers to improved production of proteins of interest. In preferred embodiments, the present invention provides enhanced (i.e., improved) production and secretion of a protein of interest. In these embodiments, the “enhanced” production is improved as compared to the normal levels of production by the host (e.g., wild-type cells). Thus, for heterologous proteins, basically any expression is enhanced, as the cells normally do not produce the protein.

As used herein, the terms “isolated” and “purified” refer to a nucleic acid or amino acid that is removed from at least one component with which it is naturally associated.

As used herein, the term “heterologous protein” refers to a protein or polypeptide that does not naturally occur in a host cell. In alternate embodiments, the protein is a commercially important industrial protein or peptide. It is intended that the term encompass protein that are encoded by naturally occurring genes, mutated genes, and/or synthetic genes.

### DETAILED DESCRIPTION OF THE INVENTION

The present invention provides means for the production of desired end-products of in vitro and/or in vivo bioconversion of biomass-based feed stock substrates, including but not limited to such materials as starch and cellulose. In particularly preferred embodiments, the methods of the present invention do not require gelatinization and/or liquefaction of the substrate.

The methods of the present invention provide means for dramatic improvements in the process for directly converting a commonly available carbon substrate such as biomass and/or starch into an intermediate, particularly intermediates that are readily convertible into a multitude of desired end-products, such as primary metabolites (e.g. ascorbic acid intermediates, lactic acid, succinic acid, or amino acids), alcohols (e.g., ethanol, propanol, and or 1,3 propanediol), and enzymes or secondary metabolites such as antimicrobials.

In some particularly preferred embodiments, the present invention provides means for dramatic improvements in processes for directly converting granular starch into glucose, an intermediate readily convertible into a multitude of desired end-products, such as primary metabolites (e.g. ascorbic acid intermediates, lactic acid, succinic acid, or amino acids), alcohols (e.g., ethanol, propanol, and or 1,3 propanediol), and enzymes or secondary metabolites such as antimicrobials.

In alternative embodiments, the present invention provides means for dramatic improvements in the process for converting starch or cellulose into glucose, which in turn is converted into the desired end-product. By maintaining the presence of the intermediate at a low concentration within the conversion media, overall efficiency of the production is improved. In some embodiments, enzymatic inhibition and/or catabolite repression, oxygen uptake demand, and/or by-product formation are reduced.

In some preferred embodiments, the maintenance of minimal intermediate concentrations is achieved by maintaining the concentration of the intermediate at a low concentration. In one embodiment, the concentration of the intermediate is less than or equal to 0.25% by weight volume of the medium (e.g., 0.25% to 0.00001% by weight volume). In other embodiments, the concentration of the intermediate is less than or equal to 0.20%, 0.10%, 0.05%, or 0.01% by weight volume (e.g., 0.20% to 0.00001%, 0.10% to 0.00001% 0.05% to 0.00001%, 0.01% to 0.00001%, respectively). Alternatively, the intermediate concentration is maintained at less than or equal to a concentration of 2.0 μmolar in the conversion media. In another embodiment, the concentration is maintained at less than or equal to 1.0 μmolar. In still another embodiment, the concentration of the intermediate is maintained at a concentration of less than or equal to 0.75 μmolar. In any event, maintaining a low concentration means maintaining the concentration of the intermediate below the threshold that results in enzyme inhibition (i.e., enzyme inhibitive effects), catabolite repression (i.e., catabolite repressive effects).

In further embodiments, the maintenance of a minimal concentration is achieved by maintaining the rate of conversion of the substrate to the intermediate at less than or equal to the rate of conversion of the intermediate to the end-product. While it is recognized that the conversion of the substrate to the intermediate is necessarily rate limiting for the conversion of the intermediate to the end-product, by providing sufficient intermediate converting enzymes for the conversion of substantially all of the intermediate produced by the first enzymatic conversion from the carbon substrate, substantially all of the intermediate is converted to the end-product as fast as it is converted from the starting substrate to minimize the presence of the intermediate in the conversion medium. Exemplary methods of providing such excessive intermediate conversion include providing an excess of intermediate converting enzyme, increasing the enzyme activity of the intermediate converting enzyme, and/or decreasing the activity of the substrate converting enzyme to convert the intermediate to end-product as quickly as it is converted from the substrate. As the actual rate of conversion is contemplated to vary with the specific end product produced, some variation in the amount and experimentation in determining the amount are contemplated. However guidelines for making these determinations are provided herein.

In some embodiments of the present invention, the conversion or consumption rate of the intermediate was determined by the calculating the amount of organism present in the mixed media, taking into consideration the other physical parameters of the mixed media, and multiplying that amount by the generally known conversion rate. This provides a rate of conversion of the intermediate, (e.g., glucose), to the end-product. In some embodiments, this conversion of the intermediate to the desired end product is by conversion or bioconversion of the intermediate to the end-product by a naturally occurring organism or one mutated to provide such bioconversion. Another embodiment of the conversion from intermediate to end product involves the use of an enzymatic conversion by a known enzyme to the desired end-product using known enzymatic conversion methods. For example, in some embodiments, the conversion of glucose to a desired end product (e.g., propanediol, succinic acid, gluconic acid, lactic acid, amino acids, antimicrobials, ethanol, ascorbic acid intermediates and/or ascorbic acid) is accomplished by the addition of an amount of an enzyme known to convert glucose to the specified end product desired.

Once the conversion rate of the intermediate to the desired end product is determined, the limit of the conversion of the carbon substrate to the intermediate can be determined in the same manner. By calculating the upper limit of the intermediate to end product conversion, the conversion rate of the carbon substrate to intermediate can be determined, the main consideration being that the intermediate concentration levels in the conversion media are maintained at a sufficiently low level to adversely effect the normally catabolite repressive/enzymatic inhibitory effects of the intermediate. In one embodiment, this is accomplished by maintaining the conversion rate of the intermediate to the end product in excess or equal to the rate of conversion of the carbon substrate to the intermediate. Thus, the present invention provides means for increasing the conversion rate to the end product, as well as means for restricting the conversion of the carbon substrate to the intermediate.

Another method for determining whether the rate of conversion of the intermediate to the end product is greater than or equal to the production of the intermediate from the carbon substrate is to measure the weight percentage of the intermediate in a reaction vessel. The amount of the intermediate present in a reaction vessel can be determined by various known methods, including, but not limited to direct or indirect measurement of the amount of intermediate present in a reaction vessel. Direct measurement can be by periodic assays of the contents within a reaction vessel, using assays known to identify the amount of intermediate and or end-product in the vessel. In addition, direct measurement of the amounts of intermediates within a reaction vessel include on-line gas, liquid and/or high performance liquid chromatography methodologies known in the art

Indirect measurement of the levels of intermediate or end-products produced can be assessed by the measurement of oxygen uptake or carbon dioxide production, using methods known in the art (e.g., by determining the oxygen uptake rate and/or the carbon evolution rate).

Substrates

The substrates of the present invention are carbon-based compounds that can be converted enzymatically to intermediate compounds. Suitable substrates include, but are not limited to processed materials that contain constituents which can be converted into sugars (e.g., cellulosic biomass, glycogen, starch and various forms thereof, such as corn starch, wheat starch, corn solids and wheat solids). During the development of the present invention good results were obtained with corn starch and wheat starch, although other sources, including starches from grains and tubers (e.g., sweet potato, potato, rice and cassava starch) also find use with the present invention. Various starches are commercially available. For example, corn starches are available from Cerestar, Sigma, and Katayama Chemical Industry Co. (Japan); wheat starches are available from Sigma; sweet potato starch is available from Wako Pure Chemical Industry Co. (Japan); and potato starch is available from Nakari Chemical Pharmaceutical Co. (Japan). A particularly useful carbon substrate is corn starch. In some embodiments of the present invention, granular starch is used in a slurry having a percentage of starch between about 20% and about 35%. Preferably, the starch is in a concentration between about 10% and about 35%. In some particularly preferred embodiments, the range for percent starch is between 30% and 32%. in addition to raw granular starch, other carbon substrate sources find use in the present invention include, but are not limited to biomass, polysaccharides, and other carbon based materials capable of being converted enzymatically to an intermediate.

Fermentable sugars can be obtained from a wide variety of sources, including lignocellulosic material. Lignocellulose material can be obtained from lignocellulosic waste products (e.g., plant residues and waste paper). Examples of suitable plant residues include but are not limited to any plant material such as stems, leaves, hulls, husks, cobs and the like, as well as corn stover, begasses, wood, wood chips, wood pulp, and sawdust. Examples of paper waste include but are not limited to discarded paper of any type (e.g., photocopy paper, computer printer paper, notebook paper, notepad paper, typewriter paper, and the like), as well as newspapers, magazines, cardboard, and paper-based packaging materials. The conditions for converting sugars to ethanol are known in the art. Generally, the temperature is between about 25° C. and 35° C (e.g., between 25° and 35°, and more particularly at 30° C.). Useful pH ranges for the conversion medium are provided between about 4.0 and 6.0, between 4.5 and 6.0, and between pH 5.5 and 5.8. However, it is not intended that the present invention be limited to any particular temperature and/or pH conditions as these conditions are dependent upon the substrate(s), enzyme(s), intermediate(s), and/or end-product(s) involved.

Enzymes

In some preferred embodiments of the present invention, enzymes that are substrate converting enzymes (i.e., enzymes that are able to first convert the carbon substrate into the carbon intermediate), and intermediate converting enzymes (i.e., enzymes that are able to convert the resulting intermediate into an intervening intermediate and/or the desired end-product) both find use in the present invention. Enzymes that find use in some embodiments of the present invention to convert a carbon substrate to an intermediate include, but are not limited to alpha-amylase, glucoamylase, starch hydrolyzing glucoamylase, and pullulanases. Enzymes that find use in the conversion of an intermediate to an end-product depend largely on the actual desired end-product. For example enzymes useful for the conversion of a sugar to 1,3-propanediol include, but are not limited to enzymes produced by E. coli and other microorganisms. For example enzymes useful for the conversion of a sugar to lactic acid include, but are not limited to those produced by Lactobacillus and Zymomonas. Enzymes useful for the conversion of a sugar to ethanol include, but are not limited to alcohol dehydrogenase and pyruvate decarboxylase. Enzymes useful for the conversion of a sugar to ascorbic acid intermediates include, but are not limited to glucose dehydrogenase, gluconic acid dehydrogenase, 2,5-diketo-D-gluconate reductase, and various other enzymes. Enzymes useful for the conversion of a sugar to gluconic acid include, but are not limited to glucose oxidase and catalase.

In some preferred embodiments, the alpha-amylase used in some methods of the present invention is generally an enzyme which effects random cleavage of alpha-(1-4) glucosidic linkages in starch. In most embodiments, the alpha-amylase is chosen from among the microbial enzymes having an E. C. number E. C. 3.2.1.1 and in particular E. C. 3.2.1.1-3. In some preferred embodiments, the alpha-amylase is a thermostable bacterial alpha-amylase. In most particularly preferred embodiments, the alpha-amylase is obtained or derived from Bacillus species. Indeed, during the development of the present invention good results were obtained using the SPEZYME® alpha-amylase obtained from Bacillus licheniformis (Genencor). In other embodiments, black-koji amylase described in methods for alcoholic fermentation from starch such as corn and cassava without precooking (Ueda et al., J. Ferment. Technol., 50:237-242 [1980]; and Ueda et al, J. Ferment. Technol., 58:237-242 [1980]) find use in the present invention.

As understood by those in the art, the quantity of alpha-amylase used in the methods of the present invention will depend on the enzymatic activity of the alpha-amylase and the rate of conversion of the generated glucose by the end-product converter. For example, generally an amount between 0.01 and 1.0 kg of SPEZYME® FRED (Genencor) is added to one metric ton of starch. In some embodiments, the enzyme is added in an amount between 0.4 to 0.6 kg,.while in other embodiments, it is added in an amount between 0.5 and 0.6 kg of SPEZYME® FRED/metric ton of starch..

In preferred embodiments of the present invention, the glucoamylase is an enzyme which removes successive glucose units from the non-reducing ends of starch. The enzyme can hydrolyze both the linear and branched glucosidic linkages of starch, amylose and amylopectin. In most embodiments, the glucoamylase used in the methods of the present invention are microbial enzymes. In some preferred embodiments, the glucoamylase is a thermostable fungal glucoamylase, such as the Aspergillus glucoamylase. Indeed, during the development of the present invention, good results were obtained using the DISTALLASE® glucoamylase derived from Aspergillus niger (Genencor). Glucoamylase preparations from Aspergillus niger have also been used without the use of precooking (See, Ueda et al, Biotechnol. Bioeng., 23:291[1981]). Three glucoamylases have been selectively separated from Aspergillus awamori var. kawachi for use in hydrolyzing starch (See, Hayashida, Agr. Biol. Chem., 39:2093-2099 [1973]). Alcoholic fermentation of sweet potato by Endomycopsis frbuligoeu glucoamylase without cooking has also been described (Saha et al., Biotechnol. Bioeng., 25:1181-1186 [1983]). Another enzyme that finds use in the present invention is glucoamylase (EC 3.2.1.3), an enzyme that hydrolyzes the alpha.-1,4-glucoside chain progressively from the non-reducing terminal end. This enzyme also hydrolyzes the alpha-1,6-glucoside chain. Glucoamylase is secreted from fungi of the genera Aspergillus, Rhizopus and Mucor also find use in the methods of the present invention. These enzymes further find use in glucose production and quantitative determination of glycogen and starch. Glucoamylase preparations obtained from E. fibuligera (IFO 0111) have been used to contact raw sweet potato starch for alcoholic fermentation (See, Saha et al., Biotechnol. Bioeng., 25:1181-1186 [1983]). One of this enzyme's major applications is as a saccharifying agent in the production of ethyl alcohol from starchy-materials. However, as with the other glucoamylases described herein, this enzyme also finds use in the methods of the present invention.

Additional glucoamylases that find use in the methods of the present invention include those obtained from the genera Rhizopus and Humicola, which are characterized as having particularly high productivity and enzymatic activity. Furthermore, in comparison with the glucoamylase derived from other organisms, the Rhizopus-derived glucoamylase exhibits a strong action on starch and its enzymological and chemical properties including optimum pH are particularly suitable for the saccharification of cereal starch. Because of these features, the Rhizopus-derived glucoamylase is considered to be best suited for alcohol production using non-cooked or low-temperature cooked starch (See, U.S. Pat. Nos. 4,514,496 and 4,092,434). It has been noted that upon the incubation of raw corn starch with Rhizopus glucoamylase, was used in conjunction with Rhizopus alpha amylase, the starch degradation-by glucoamylase was accelerated. While it is not intended that the present invention be limited to any particular mechanism or theory, it is believed that Rhizopus glucoamylase has a stronger degradation activity than Aspergillus niger glucoamylase preparations which also contain α-amylase (See, Yamamoto et al., Denpun Kagaku, 37:129-136 [1990]). One commercial preparation that finds use in the present invention is the glucoamylase preparation derived from the Koji culture of a strain of Rhizopus niveus available from Shin Nippo Chemical Co., Ltd. Another commercial preparation that finds use in the present invention is the commercial starch hydrolyzing composition M1 is available from Biocon India, of Bangalore, India.

As understood by those in the art, the quantity of glucoamylase used in the methods of the present invention depends on the enzymatic activity of the glucoamylase (e.g., DISTILLASE® L-400). Generally, an amount between 0.001 and 2.0 ml of a solution of the glucoamylase is added to 450 gm of a slurry adjusted to 20-35% dry solids, the slurry being the liquefied mash during the saccharffication and/or in the hydrolyzed starch and sugars during the fermentation. In some embodiments, the glucoamylase is added in an amount between 0.005 and 1.5 ml of such a solution. In some preferred embodiments, the enzyme is added at an amount between 0.01 and 1.0 ml of such a solution.

As indicated above, pullulanases also find use in the methods of the present invention. These enzymes hydrolyze alpha.-1,6-glucosidic bonds. Thus, during the saccharification of the liquefied starch, pullulanases remove successive glucose units from the non-reducing ends of the starch. This enzyme is capable of hydrolyzing both the linear and branched glucosidic linkages of starch, amylose and amylopectin.

Additional enzymes that find use in the present invention include starch hydrolyzing (RSH) enzymes, including Humicola RSH glucoamylase enzyme preparation (See, U.S. Pat. No. 4,618,579). This Humicola RSH enzyme preparation exhibits maximum activity within the pH range of 5.0 to 7.0 and particularly in the range of 5.5 to 6.0. In addition, this enzyme preparation exhibits maximum activity in the temperature range of 50° C. to 60° C. Thus, in each of the steps of the present invention in which this enzyme is used, the enzymatic solubilization of starch is preferably carried out within these pH and temperature ranges.

In some embodiments, Humicola RSH enzyme preparations obtained from the fungal organism strain Humicola grisea var. thermoidea find use in the methods of the present invention. In some particularly preferred embodiments, these Humicola RSH enzymes are selected from the group consisting of ATCC (American Type Culture Collection) 16453, NRRL (USDA Northern Regional Research Laboratory) 15219, NRRL 15220, NRRL 15221, NRRL 15222, NRRL 15223, NRRL 15224, and NRRL 15225, as well as genetically altered strains derived from these enzymes.

Additional RSH glucoamylases that find use in the methods of the present invention include Rhizopus RSH glucoamylase enzyme preparations. In some embodiments, the enzyme obtained from the Koji strain of Rhizopus niveus available from Shin Nihon Chemical Co., Ltd., Ahjyo, Japan, under the tradename “CU CONC” is used. Another useful enzyme preparation is a commercial digestive from Rhizopus available from Amano Pharmaceutical under the tradename “GLUCZYME” (See, Takahashi et al., J. Biochem., 98:663-671 [1985]). Additional enzymes include three forms of glucoamylase (EC 3.2.1.3) of a Rhizopus sp., namely “Gluc1” (MW 74,000), “Gluc2” (MW 58,600) and “Gluc 3” (MW 61,400). Gluc1 was found to be 22-25 times more effective than Gluc2 or Gluc3. Thus, although Gluc2 and Gluc3 find use in the present invention, because Gluc1 tightly binds to starch and has an optimum pH of 4.5, Gluc1 finds particular use in the present invention. An additional RSH glucoamylase enzyme preparation for use in the present invention includes enzyme preparations sold under the designation “M1,” available from Biocon India, Ltd., Bangalore, India (M1 is a multifaceted enzyme composition or mixture).

As noted above, in most embodiments, Humicola RSH glucoamylase enzyme preparations contain glucoamylase activity as well as a potentiating factor which solubilizes starch. The relative proportions of potentiating factor and glucoamylase activity in other RSH enzyme preparations may vary somewhat. However, with RSH glucoamylase enzyme preparations that find use in the present invention, there is usually ample potentiating factor produced along with the glucoamylase fraction. Accordingly, the activity of the RSH glucoamylase enzyme preparations is defined in terms of their glucoamylase activity.

In addition to the use of enzymatic compositions containing the above described hydrolyzing enzymes, the present invention provides methods in which a microorganism is exposed to a substrate and uses the substrate to produce the desired end-product. Thus, in some embodiments, contacting the substrate or intermediate with a fungal, bacterial or other microorganism that produces the desired end-product is used to convert the substrate or intermediate to the desired intermediate or end-product. For example, Lactobacillus amylovorous (ATCC 33621) is a lactic acid producing bacteria isolated from cattle manure corn enrichments (See, Nkamura, Int. J. Syst. Bacteriol., 5 31:56-63 [1981]). This strain produces an extracellular amylase which enables it to hydrolyze liquefied (soluble) starch to glucose, which can then be fermented to lactic acid. (See, Xiaodong et al., Biotechnol. Lett., 19:841-843 [1997]). E. coli produces 1,3-propanediol and succinic acid, which can be contacted with glucose to produce glycerol and 1,3-propanediol.

Indeed, commercially available alpha-amylases and glucoamylases find use in the methods of the present invention in economically realistic enzyme concentrations. Although commonly used fermentation conditions do not utilize optimum temperatures, the pH conditions for fermentation do correspond closely to the optimum pH for commercially available saccharification enzymes (i.e., the glucoamylases). In some. embodiments of the present invention, complete saccharification to glucose is favored by the gradual solubilization of granular starch. Presumably, the enzyme is always exposed to low concentrations of dextrin. In addition, the removal of glucose throughout the fermentation maintains a low glucose content in the fermentation medium. Thus, glucoamylase is exposed to low concentration of glucose. In consequence, the glucoamylase is used so effectively that economically feasible dosage levels of glucoamylase (GAU) are suitable for use in the methods of the present invention (i.e.,. glucoamylase dosage of 0.05-10.0 GAU/g of starch; and preferably 0.2-2.0 GAU/g starch).

The dosages provided above for glucoamylase only approximate the effective concentration of the enzymatic saccharification activity in the fermentation broth, as an additional proportion of the saccharification activity is contributed by the alpha-amylase. Although it is not intended that the present invention be limited to any particular mechanism or theory, it is believed that the alpha-amylase further widens the holes bored by glucoamylase on starch granules (See, Yamamoto et al., supra). Typically, the use of commercially available alpha-amylases results in the production of significant amounts of sugars, such as glucose and maltose.

It is contemplated that addition of the alpha-amylase from Aspergillus oryzae (e.g., CLARASE® L (Genencor International Inc.) to wort will find use in the brewing industry. This particular enzyme saccharifies dextrins to maltotriose and maltose. Thus, although the purpose of the alpha-amylase is to liquefy starch, its saccharification propensity also make the alpha-amylase a portion of the saccharifying enzyme content.

Furthermore, some commercially available glucoamylases contain some alpha-amylase activity. Thus, it is possible (albeit usually not practical) to ferment particulate starch in the presence solely of glucoamylase. However, it is not intended that such embodiments be excluded from the present invention.

In most embodiments of the methods of the present invention, an effective amount of alpha-amylase is added to a slurry of particulate starch. Those of skill in the art understand that in addition to the uncertain amount of alpha-amylase activity contributed by glucoamylase, the effective activity of the alpha-amylase may be quite different from the unit activity values given by the supplier. The activity of alpha-amylase is pH dependent, and may be different at the pH range selected for the fermentation (i.e., as compared with the test conditions employed by the suppliers for their reported unit activity values). Thus, some preliminary experiments are contemplated as being sometimes necessary in order establish the most effective dosages for alpha-amylases, including those not explicitly described herein, but find use in the methods of the present invention.

In some most preferred embodiments, the alpha-amylase dosage range for fungal alpha-amylases is from 0.02 SKBU/g (Fungal Alpha Amylase Units) to 2.0 SKBU/g of starch, although in some particularly preferred embodiments, the range is 0.05-0.6 SKBU/g. One “SKBU” is as known in the art (See, Cerial Chem., 16:712-723 [1939]). In most embodiments utilizing Bacillus alpha-amylases, the range is 0.01 LU/g to 0.6 LU/g, preferably 0.05 to 0.15 LU/g. It is contemplated that the uncertainty as to the real activity of both the glucoamylase and the alpha-amylase in the fermenting slurry will require some preliminary investigation into the practice of some embodiments. Optimization considerations include the fact that increasing the alpha-amylase dosage with a constant glucoamylase content, increases the fermentation rate. In addition, increasing the glucoamylase dosage with a constant alpha-amylase content increases the fermentation rate. Holding the dosage of enzyme constant and/or increasing the starch content in the slurry also increase the fermentation rate. Indeed, it is contemplated that in some embodiments, the optimum alpha-amylase dosage well exceeds dosages heretofore recommended for liquefying starch; the optimum glucoamylase may well exceed dosages recommended for saccharifying syrups. However, enzyme dosage levels should not be confused with enzyme usage. Substantial proportions of the enzymes dosed into the starch slurry are available for recovery from the fermentation broth for use anew to ferment granular starch.

A further consideration arising from employment of the enzymes at fermentation temperatures is that although the enzymes exhibit low relative activity (e.g., activity of the alpha-amylase from B. licheniformis at fermentation temperatures is not more than about 25% of maximum activity), the low relative activity is counterbalanced by the extended duration of the 48-120 hours of fermentation, and by the extended half-life of enzymes that have not been subjected to elevated temperatures. Indeed, it has been determined that more than 90% of the enzymes activity remains after 72 hours of fermentation. The alpha-amylase of B. licheniformis (SPEZYME® AA and SPEZYME® FRED enzymes; Genencor International Inc.) is sufficiently stable to withstand brief exposures to still pot temperatures. Thus, recycle of stillage can be used as a way to recycle alpha-amylase. However, recovery of enzyme in recycled stillage requires care, in avoiding subjecting the fermentation broth to ethanol stripping temperatures that deactivate the enzyme(s). For example, the alcohol may be vacuum stripped from the fermentation broth and such stillage recycled to recover the enzymes suitable for use in subsequent reactions.

However, as earlier described, some RSHs glucoamylases (e.g., the enzyme obtained from Rhizopus) are available that convert starch to glucose at non-cooking temperatures, reducing the need for exposing the enzymatic composition to still pot temperatures. This reduces the energy costs of converting the carbon substrate to the desired end-product, thereby reducing the overall costs of manufacturing. Thus, these enzymes find particular use in the methods of the present invention.

In preferred embodiments of the present invention, once the carbon source is enzymatically converted to the intermediate, it is converted into the desired end-product by the appropriate methodology. Conversion is accomplished via any suitable method (e.g., enzymatic or chemical). In one preferred embodiment, conversion is accomplished by bioconversion of the intermediate by contacting the intermediate with a microorganism. In alternate preferred embodiments, the respective substrate-converting enzyme and the intermediate-converting enzyme are placed in direct contact with the substrate and/or intermediate. In some embodiments, the enzyme(s) are provided as isolated, purified or concentrated preparations.

In further embodiments, the substrate and/or intermediate are placed in direct contact with a microorganism (e.g., bacterium or fungus) that secretes or metabolizes the respective substrate or intermediate. Thus, the present invention provides means for the bioconversion of a substrate to an end-product. In some embodiments, at least one intermediate compound is produced during this conversion process.

In some embodiments, microorganisms that are genetically modified to express enzymes not normally produced by the wild-type organism are utilized. In some particularly preferred embodiments, the organisms are modified to overexpress enzymes that are normally produced by the wild-type organism.

The desired end-product can be any product that may be produced by the enzymatic conversion of the substrate to the end-product. In some preferred embodiments, the substrate is first converted to at least one intermediate and then converted from the intermediate to an end-product. For example, hexoses can be bioconverted into numerous products, such as ascorbic acid intermediates, ethanol, 1,3-propanediol, and gluconic acid. Ascorbic acid intermediates include but are not limited to 2,5-diketoglucbnate, 2 KLG (2-keto-L-gluconate), and 5-KDG (5-keto-D-gluconate). Gluconate can be converted from glucose by contacting glucose with glucose dehydrogenase (GDH). In addition, gluconate itself can be converted to 2-KDG (2-keto-D-gluconate) by contacting gluconate with GDH. Furthermore, 2-KDG can be converted to 2,5-DKG by contacting 2-KDG with 2-KDGH. Gluconate can also be converted to 2-KDG by contacting gluconate with 2KR. Glucose can also be converted to 1,3-propanediol by contacting glucose with E. coli. In addition, glucose can be converted to succinic acid by contacting glucose with E. coli. Additional embodiments, as described herein are also provided by the present invention.

In some embodiments in which glucose is an intermediate, it is converted to ethanol by contacting glucose with an ethanologenic microorganism. In contacting the intermediate with an intermediate converting enzyme, it is contemplated that isolated and/purified enzymes are placed into contact with the intermediate. In yet another embodiment, the intermediate is contacted with bioconverting agents such as bacteria, fungi or other organism that takes in the intermediate and produces the desired end-product. In some embodiments, the organism is wild-type, while in other embodiments it is mutated.

Preferred examples of ethanologenic microorganisms include ethanologenic bacteria expressing alcohol dehydrogenase and pyruvate decarboxylase, such as can be obtained with or from Zymomonas mobilis (See e.g., U.S. Pat. Nos. 5,028,539, 5,000,000, 5,424,202, 5,487,989, 5,482,846, 5,554,520, 5,514,583, and copending applications having U.S. Ser. No. 08/363,868 filed on Dec. 27, 1994, U.S. Ser. No. 08/475,925 filed on Jun. 7, 1995, and U.S. Ser. No. 08/218,914 filed on Mar. 28, 1994.

In additional embodiments, the ethanologenic microorganism expresses xylose reductase and xylitol dehydrogenase, enzymes that convert xylose to xylulose. In further embodiments, xylose isomerase is used to convert xylose to xylulose. In additional embodiments, the ethanologenic microorganism also expresses xylulokinase, an enzyme that catalyzes the conversion of xylulose to xylulose-5-phosphate. Additional enzymes involved in the completion of the pathway include transaldolase and transketolase. These enzymes can be obtained or derived from Escherichia coli, Klebsiella oxytoca and Erwinia species (See e.g., U.S. Pat. No. 5,514,583).

In some particularly preferred embodiments, a microorganism capable of fermenting both pentoses and hexoses to ethanol are utilized. For example in some embodiments, a recombinant organism which inherently possesses one set of enzymes and which is genetically engineered to contain a complementing set of enzymes is used (See e.g., U.S. Pat. Nos. 5,000,000, 5,028,539, 5,424,202, 5,482,846, 5,514,583, and WO 95/13362). In some embodiments, particularly preferred microorganisms include Klebsiella oxytoca P2 and E. coli Ko11.

In some embodiments, supplements are added to the nutrient medium (i.e., the culture medium), including, but not limited to vitamins, macronutrients, and micronutrients. Vitamins include, but are not limited to choline chloride, nicotinic acid, thiamine HCl, cyanocobalamin, p-aminobenzoic acid, biotin, calcium pantothenate, folic acid, pyridoxine.HCl, and riboflavin. Macronutrients include, but are not limited to (NH₄)₂SO₄, K₂HPO₄, NaCl, and MgSO₄.7H₂O. Micronutrients include, but are not limited to FeCl₃ 6H₂O, ZnCl₂.4H₂O, CoCl₂.6H₂O, molybdic acid (tech), CuCl₃. and H₃BO₃.

Media and Carbon Substrates

The conversion media in the present invention must contain suitable carbon substrates. Suitable carbon substrates include, but are not limited to biomass, monosaccharides (e.g., glucose and fructose), disaccharides (e.g., lactose and sucrose), oligosaccharides, polysaccharides (e.g., starch and cellulose), as well as mixtures thereof, and unpurified mixtures from renewable feedstocks such as cheese whey permeate, cornsteep liquor, sugar beet molasses, and barley malt. In additional embodiments, the carbon substrate comprises one-carbon substrates such as carbon dioxide, or methanol for which metabolic conversion into key biochemical intermediates has been demonstrated.

Glycerol production from single carbon sources (e.g., methanol, formaldehyde or formate) has been reported in methylotrophic yeasts (See, Yamada et al., Agric. Biol. Chem., 53:541-543 [1989]) and in bacteria (Hunter et.al., Biochem., 24:4148-4155 [1985]). These organisms can assimilate single carbon compounds, ranging in oxidation state from methane to formate, and produce glycerol. In some embodiments, the pathway of carbon assimilation is through ribulose monophosphate, through serine, or through xylulose-monophosphate (See e.g., Gottschalk, Bacterial Metabolism, 2nd Ed., Springer-Verlag, New York [1986]). The ribulose monophosphate pathway involves the condensation of formate with ribulose-5-phosphate to form a 6-carbon sugar that becomes fructose and eventually the 3-carbon product glyceraldehyde-3-phosphate. Likewise, the serine pathway assimilates the one-carbon compound into the glycolytic pathway via methylenetetrahydrofolate.

In addition to the utilization of one and two carbon substrates, methylotrophic organisms are also known to utilize a number of other carbon-containing compounds such as methylamine, glucosamine and a variety of amino acids for metabolic activity. For example, methylotrophic yeast are known to utilize the carbon from methylamine to form trehalose or glycerol (Bellion et al., in Murrell et al. (eds), 7^{th} Microb. Growth C1 Compd., Int. Symp., 415-32, Intercept, Andover, UK [1993]). Similarly, various species of Candida metabolize alanine or oleic acid (Sulter et al., Arch. Microbiol., 153:485-9 [1990]). Hence, the source of carbon utilized in the present invention encompasses a wide variety of carbon-containing substrates and is only limited by the requirements of the host organism.

Although it is contemplated that all of the above mentioned carbon substrates and mixtures thereof will find use in the methods of the present invention, preferred carbon substrates include monosaccharides, disaccharides, oligosaccharides, polysaccharides, and one-carbon substrates. In more particularly preferred embodiments, the carbon substrates are selected from the group consisting of glucose, fructose, sucrose and single carbon substrates such as methanol and carbon dioxide. In a most particularly preferred embodiment, the substrate is glucose.

As known in the art, in addition to an appropriate carbon source, fermentation media must contain suitable nitrogen source(s), minerals, salts, cofactors, buffers and other components suitable for the growth of the cultures and promotion of the enzymatic pathway necessary for the production of the desired end-product (e.g., glycerol). In some embodiments, (II) salts and/or vitamin B₁₂ or precursors thereof find use in the present invention.

Culture Conditions

Typically, cells are grown at approximately 30° C. in appropriate media. Preferred growth media utilized in the present invention include common commercially prepared media such as Luria Bertani (LB) broth, Sabouraud Dextrose (SD) broth or Yeast Malt Extract (YM) broth. However, other defined or synthetic growth media may also be used, as appropriate. Appropriate culture conditions are well-known to those in the art.

In some embodiments, agents known to modulate catabolite repression directly or indirectly (e.g., cyclic adenosine 2′:3′-monophosphate or cyclic. adenosine 2′:5′-monophosphate), are incorporated into the reaction media. Similarly, the use of agents known to modulate enzymatic activities (e.g., sulphites, bisulphites and alkalis) that lead to enhancement of glycerol production also find use in conjunction with or as an alternative to genetic manipulations.

Suitable pH ranges for fermentation are between pH 5.0 to pH 9.0; while the range of pH 6.0 to pH 8.0 is particularly preferred for the initial conditions of the reaction system. Furthermore, reactions may be performed under aerobic, microaerophilic, or anaerobic conditions, as suited for the organism utilized.

Batch and Continuous Fermentations

In some preferred embodiments, the present process uses a batch method of fermentation. A classical batch fermentation is a closed system, wherein the composition of the media is set at the beginning of the fermentation and is not subject to artificial alterations during the fermentation. Thus, at the beginning of the fermentation the medium is inoculated with the desired organism(s). In this method, fermentation is permitted to occur without the addition of any components to the system. Typically, a batch fermentation qualifies as a “batch” with respect to the addition of the carbon source and attempts are often made at controlling factors such as pH and oxygen concentration. The metabolite and biomass compositions of the batch system change constantly up to the time the fermentation is stopped. Within batch cultures, cells moderate through a static lag phase to a high growth log phase and finally to a stationary phase where growth rate is diminished or halted. If untreated, cells in the stationary phase eventually die. In general, cells in log phase are responsible for the bulk of production of end product or intermediate.

A variation on the standard batch system is the “fed-batch fermentation” system, which also finds use with the present invention. In this variation of a typical batch. system, the substrate is added in increments as the fermentation progresses. Fed-batch systems are useful when catabolite repression is apt to inhibit the metabolism of the cells and where it is desirable to have limited amounts of substrate in the media. Measurement of the actual substrate concentration in fed-batch systems is difficult and is therefore estimated on the basis of the changes of measurable factors such as pH, dissolved oxygen and the partial pressure of waste gases such as CO₂. Batch and fed-batch fermentations are common and well known in the art.

It is also contemplated that the methods of the present invention are adaptable to continuous fermentation methods. Continuous fermentation is an open system where a defined fermentation media is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth and/or end product concentration. For example, in one embodiment, a limiting nutrient such as the carbon source or nitrogen level is maintained at a fixed rate an all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to media being drawn off must be balanced against the cell growth rate in the fermentation. Methods of modulating nutrients and growth factors for continuous fermentation processes as well as techniques for maximizing the rate of product formation are well known in the art of industrial microbiology.

In some embodiments, the present invention is practiced using batch processes, while in other embodiments, fed-batch or continuous processes, as well as any other suitable mode of fermentation are used. Additionally, in some embodiments, cells are immobilized on a substrate as whole-cell catalysts and are subjected to fermentation conditions for the appropriate end-product production.

Alterations in the Enzymatic Pathway

Various alterations in enzymatic pathways are contemplated for use in the methods of the present invention. One representative enzyme pathway involves he production of 1,3-propanediol from glucose. In some embodiments, this is accomplished by the following series of steps which are representative of a number of pathways known to those skilled in the art. In this representative pathway, glucose is converted through a series of steps by enzymes of the glycolytic pathway to dihydroxyacetone phosphate (DHAP) and 3-phosphoglyceraldehyde (3-PG). Glycerol is then formed by either hydrolysis of DHAP to dihydroxyacetone (DHA) followed by reduction, or reduction of DHAP to glycerol 3-phosphate (G3P) followed by hydrolysis. The hydrolysis step can be catalyzed by any number of cellular phosphatases which are known to be specific or non-specific with respect to their substrates or the activity can be introduced into the host by recombination. In some embodiments, the reduction step is catalyzed by a NAD⁺ (or NADP⁺)-linked host enzyme or the activity is introduced into the host by recombination. It is noted that the dha regulon contains a glycerol dehydrogenase (E.C. 1.1.1.6) which catalyzes the reversible reaction of Equation 3.

Glycerol→3-HP+H₂O (Equation 1)

3-HP+NADH+H⁺→1,3-Propanediol+NAD⁺ (Equation 2)

Glycerol+NAD⁺→DHA+NADH+H⁺ (Equation 3)

Glycerol is converted to 1,3-propanediol via the intermediate 3-hydroxypropionaldehye (3-HP) as has been described in detail above. The intermediate 3-HP is produced from glycerol (Equation 1) by a dehydratase enzyme which can be encoded by the host or can introduced into the host by recombination. This dehydratase can be glycerol dehydratase (E.C. 4.2.1.30), diol dehydratase (E.C. 4.2.1.28), or any other enzyme able to catalyze this transformation. Glycerol dehydratase, but not diol dehydratase, is encoded by the dha regulon. In some embodiments, 1,3-propanediol is produced from 3-HP (Equation 2) by a NAD⁺ or NADP⁺ linked host enzyme, while in other embodiments, the activity is introduced into the host by recombination. In some embodiments ,this final reaction in the production of 1,3-propanediol is catalyzed by 1,3-propanediol dehydrogenase (E.C. 1.1.1.202) or other alcohol dehydrogenases. It is noted that in some embodiments, mutations and transformations affect carbon channeling. A variety of mutant organisms comprising variations in the 1,3-propanediol production pathway find use in the present invention. The introduction of a triosephosphate isomerase mutation (tpi-) into the microorganism is an example of the use of a mutation to improve the performance by carbon channeling. Alternatively, mutations which diminish the production of ethanol (adh) or lactate (ldh) increase the availability of NADH for the production of 1,3-propanediol. Additional mutations in steps of glycolysis after glyceraldehyde-3-phosphate include the 1,3-propanediol production pathway. Mutations that effect glucose transport such as PTS which would prevent loss of PEP also find use in the present invention. Mutations which block alternate pathways for intermediates of the 1,3-propanediol production pathway such as the glycerol catabolic pathway (glp) also find use in the present invention. In some embodiments, the mutation is directed toward a structural gene, so as to impair or improve the activity of an enzymatic activity or can be directed toward a regulatory gene so as to modulate the expression level of an enzymatic activity.

In additional embodiments, transformations and mutations are combined to as to control particular enzyme activities for the enhancement of1,3-propanediol production. Thus, it is within the scope of the present invention to provide modifications of a whole cell catalyst which lead to an increased production of 1,3-propanediol.

Identification and Purification of the End-Product

Methods for the purification of the end-product from fermentation media are known in the art. For example, propanediols can be obtained from cell media by subjecting the reaction mixture to extraction with an organic solvent, distillation and column chromatography (See e.g., U.S. Pat. No. 5,356,812). A particularly good organic solvent for this process is cyclohexane (See, U.S. Pat. No. 5,008,473).

In some embodiments, the end-product is identified directly by submitting the media to high pressure liquid chromatography (HPLC) analysis. One method of the present invention involves analysis of fermentation media on an analytical ion exchange column using a mobile phase of 0.01 N sulfuric acid in an isocratic fashion.

Identification and Purification of the Enzymes

The enzyme levels in the media can be measured by enzyme assays. For example in the manufacture of 1,3-propanediol, the levels of expression of the proteins G3PDH and G3P phosphatase are measured by enzyme assays. The G3PDH activity assay relies on the spectral properties of the cosubstrate, NADH, in the DHAP conversion to G-3-P. NADH has intrinsic UV/vis absorption and its consumption can be monitored spectrophotometrically at 340 nm. G3P phosphatase activity can be measured by any method of measuring the inorganic phosphate liberated in the reaction. The most commonly used detection method used the visible spectroscopic determination of a blue-colored phosphomolybdate ammonium complex.

Thus, although there are various superficial resemblances between the methods known in the art and the methods of the present invention, the present invention provides more comprehensive objectives that are reflected in a great number of detail features believed to be unique to practice of this invention, including notably enzyme recycling, biomass and starch recycling.

Recovery

Overall, recovery of enzymes in recycled stillage requires care, in order to avoid subjecting the conversion media to temperatures that deactivate the enzymes. In one example, for the recovery of ethanol, the alcohol is vacuum stripped from the fermentation broth and the stillage is recycled, in order to recover the enzymes. In some embodiments, enzymes are recovered through the use of ultrafiltration or-an electrodialysis device and recycled.

Process Considerations

As indicated above, fermentation of granular starch slurry has completely different characteristics than fermentation of a syrup. Generally, a concentration of about 20% solids in solution is considered the maximum sugar content in a fermentation medium, as higher concentrations create difficulties at the onset and at the end of fermentation. However, no similar limits exist in the fermentation of a starch slurry. The concentration of starch in the slurry may vary from 10-35 %, with no discernable consequences at the onset of fermentation. Increasing starch concentration (e.g., at constant enzyme dosages) speeds up the bioconversion rate, or conversely, allows for lowering the enzyme dosages required to achieve a given bioconversion rate. The excess (i.e., residual) granular starch may be recovered, along with substantial amounts of enzymes and subjected to renewed fermentation. Thus, control over starch concentration is a major process parameter for practice of this invention.

In one preferred embodiment, means for bioconversion and fermentation of a granular starch slurry having 10-35% starch by weight are provided. In some preferred embodiments, fermentation of a 10-35% starch slurry with E. coli results in the production of residual starch when fermentation has proceeded to the intended organic acid or 1,3-propane diol content levels. However, this reaction is dependent on the microorganism and bioprocessing conditions used and, therefore, recycling of the enzymes on the starch particles occurs when the residual starch is again fermented. However, even when a 25-35% starch slurry is fermented, in preferred embodiments, the fermentation is halted before complete disappearance of the granular starch, for fermentation anew. Thus, recycling of starch is a facile way to recover enzymes for reuse.

In one preferred embodiment of the present invention, the (granular) starch and microorganisms are removed together (e.g., by centrifugation or filtration). This removed starch and microorganisms are mixed with fresh granular starch and additional aliquot(s) of enzyme(s) as needed, to produce a fermentation charge for another fermentation run.

In another preferred embodiment, bioconversion and fermentation of a corn stover slurry having 10-25% cellulosics by weight is provided. In one embodiment, fermenting a 10-25% cellulosic slurry with E. coli results in residual cellulosics when fermentation has proceeded to the intended organic acid or 1.,3-propane diol content levels. This reaction is dependent upon the microorganism and bioprocessing conditions used. As above, recycling of the enzymes on the cellulosics occurs when the residual corn stover is again fermented. However, even when a 25-35% cellulosics slurry is fermented, in some preferred embodiments, the fermentation is halted before the complete disappearance of the stover, for fermentation anew. Thus, recycling stover is a facile way to recover enzymes for reuse.

In yet another preferred embodiment, the corn stover and microorganisms are removed together (e.g., by centrifugation or filtration). This removed corn stover and microorganisms are mixed with fresh corn stover and additional aliquot(s) of enzyme(s) as needed, to produce a fermentation charge for another fermentation run.

As recognized by those of skill in the art, engineering trade-offs are contemplated in arriving at optimum process details; these trade-offs are contemplated to vary, depending upon each particular situation. Nonetheless, the methods provided herein provide the necessary teachings to make such trade-offs to obtain optimum processes. For example, to achieve the most rapid fermentation reasonable, high starch or cellulose content, and high enzymes dosage are indicated. But, the consequential rapid fermentation tails off into generation of a level of nutrients in the fermentation broth, when then dictates recovery of the nutrients, or, alternatively that fermentation be halted at a relatively low end-product (e.g., alcohol) content. However, in situations where relatively low fermentation rates are acceptable, then (with high starch content slurries) enzyme dosage is relatively low and nutrient losses are held to levels heretofore accepted by the fermentation arts. In cases where maximum yield of end-product (e.g., alcohol) is a principal objective, then low starch content slurries, moderate alpha-amylase dosage, and high glucoamylase dosage find use in the present invention. However, it is not intended that the present invention be limited to any particular method design.

As indicated herein, the present invention saves considerable thermal energy. However, just as the starting substrate (e.g., starch) is never subjected to the thermal conditions used for liquefactions, the substrate is not thermally sterilized. Thus, it is contemplated that is some embodiments, the starting substrate (e.g., granular starch) adds contaminating microorganisms to the fermentation medium. Thus, in some embodiments, it is advantageous to seed the fermentation medium with a large number of the product-producing microorganisms that are associated with recycled substrate (e.g., starch). By greatly outnumbering the contaminants, the recycled microorganisms overwhelm any contaminating microorganisms, thereby dominating the fermentation, resulting in the production of the desired end-product.

In some embodiments, the quantities of microorganisms and/or enzymes initially charged into the fermentation vat or bioreactor are in accord with prior art practices for the fermentation and/or bioconversion of various products. These quantities will vary, as the microbial cells multiply during the course of the fermentation whereas enzymes used for bioconversion will have a limited half-life. Although in some embodiments, recycling of microorganisms is utilized, in many embodiments, it is not required for the practice of the present invention. In contrast, in particularly preferred embodiments, it is desirable to recycle enzymes (although it is not intended that the present invention be limited to is methods which require the recycling of enzymes).

Thus, in some embodiments, removal of the microbes from the residual starch or biomass particles prior to recycling of the residual starch or biomass is contemplated. However, it is again noted that practice of the present invention does not necessarily require thermal treatment of the starting substrate (e.g., starch). Thus, in some embodiments, the starting substrate is heat-sterilized, while in other embodiments, it is not. Therefore, in some embodiments, the fermentation/bioconversion is conducted in the presence of a relatively large proportion of microorganisms, in order to overcome the effects of any contamination. In alternative embodiments, antimicrobials are added to the fermentation medium to suppress growth of contaminating microorganisms. In additional embodiments, cold sterilization techniques, UV radiation, 65° C. pasteurization are used to sterilize the starting (e.g,, substrate) materials. However, biomass poses no problem regarding sterilization of fermentation vats or bioreactors.

As described herein, the present invention provides means to control the fermentation rate by releasing metabolizable sugars to the microbes or to subsequent enzymes at a controlled rate. The methods of the present invention are very different from what has been done heretofore. The prior art teaches the treatment of solid starch with enzymes prior to fermentation and/or inclusion of enzymes in the fermentation medium to conserve energy and/or to improve fermentation efficiency. However, in contrast to the present invention, there is no teaching in the art to alter the character of the fermentation so as to achieve a near to linear fermentation rate. The present invention provides means to efficiently conserve energy, particularly as compared to high temperature starch liquefaction. Indeed, in preferred embodiments, more thermal energy is conserved. The methods of the present invention operate with high fermentation efficiency, in part because product losses due to starch retrogradation, incomplete saccharification, and incomplete fermentation of fermentables are reduced. Furthermore, the ability to tailor the fermentation rate through control of starch or biomass concentration, as well as controlling the enzyme content and proportions, as provided by the present invention, facilitates the production of the desired end-products with minimal carbohydrate content.

As further indicated in the following Examples, the present invention provides novel methods for the production of gluconic acid using enzymatic conversion of starch. As indicated, using this enzymatic conversion of starch to gluconate helps remove two significant barriers currently encountered during the production of gluconate form glucose using enzymes. To compete with current gluconic acid production process, glucose needed to be used in 30-60 wt % solution, which partially inhibits glucose oxidase/catalase enzyme system at concentrations that high. In presently used methods, glucose concentrations this high result in a very high dosage of these enzymes and thus make the process economically prohibitive. An additional problem of currently used methods is that with use of 60% sugar solution substrates, there is a high viscosity level which negatively impacts solubility of oxygen in the reaction mixture. Oxygen is the second substrate and is required equimolarly for this oxidation. Lower availability of oxygen in the solution leads to lower rate of oxidation of glucose to gluconic acid and thus requires better Kla (oxygen delivering constant) delivering reactors.

Use of starch as the starting material does not only address the above shortcomings of currently used methods, but has at least three additional significant benefits in terms of the raw material cost of corn starch vs. D-glucose, reduction of substrate and/or product-based inhibition of enzymes employed in the bioconversion, and a concomitant significant reduction in the requirement of high enzyme dosage(s) for the production of gluconic acid.

In sum, the present invention provides novel methods for the production of gluconate from raw corn starch. Indeed, the present invention provides the first demonstration of the conversion of starch to gluconic acid using in vitro bioreactor and enzymatic bioconversion. The methods of the present invention further provide means for using a lower-cost renewable feed stock for the formation of a key commodity, namely industrial chemical gluconic acid.

In addition, the following Examples demonstrate that maltodextrin can be efficiently converted to gluconate using Genencor's OPTIMAX®, OXYGO® and FERMCOLASE® enzymes. It is also demonstrated that by using an optimized ratio of enzymes, the damaging effects-of hydrogen peroxide produced during the reaction can be circumvented. In addition, the following Examples indicate that It is also possible to maintain the requisite dissolved oxygen requirement in the reactor for the oxidation of glucose produced from maltodextrin by configuring the enzyme dosages of all the three enzymes. It is also demonstrated that by optimizing the dosage of OPTIMAX (alpha amylase; Genencor), it is possible to control the release of glucose in the reaction mixture.

Furthermore, the following Examples demonstrate that fermentation control via alternate and cheaper carbon-feed stocks like starch, and biomass using enzyme-based conversion offers a more economical and efficient, as well as sustainable fermentation strategy to produce industrial chemicals, enzymes and therapeutics. As indicated in the following Examples, the rate of glucose release is controllable by the amount of enzyme addition. Indeed, it was observed that rate of starch conversion using glucoamylase was 100 fold faster than was initially predicted. However, the rate of glucose conversion to product is dependent upon the available glucose concentration in the medium and thus effects the final product formation. Thus, by controlling the release of glucose for available conversion by the amount of glucoamylase added, a means for manipulating the reaction to provide the fastest conversion rate achievable for product formation is provided.

In addition, the selectivity of conversion is controllable based on the dosage of glucoamylase used. As indicated in the following Examples, the best rate of product formation was produced using 3 units of enzymes. However, it is contemplated that the user of the present invention will modify the exact reaction conditions to suit their particular needs. Indeed, the details of each process are contemplated to vary, depending upon the kinetics of hydrolyzing enzymes used and the kinetics of glucose to product conversion. In addition, external reaction condition, such as pH, temp, and medium formulation are likewise important considerations. Nonetheless, the present invention provides the teachings necessary for the practice of the present invention under various conditions.

It is also contemplated that the methods of the present invention for efficient conversion of carbon feedstocks will find use in various other fermentations, including but not limited to the efficient production bioproducts from cellulose and/or hemicellulose. It is also contemplated that the starting materials provided herein will find use as substitutes for lactose in various fermentation processes. Thus, it is contemplated that the present invention will find wide-spread use in the industrial fermentation industry.

Various other examples and modifications of the description and Examples are apparent to a person skilled in the art after reading the disclosure without departing from the spirit and scope of the invention; it is intended that all such examples or modifications be included within the scope of the appended claims. All publications and patents referenced herein are hereby incorporated by reference in their entirety.

Experimental

The following examples are provided in order to demonstrate and further illustrate certain preferred embodiments and aspects of the present invention and are not to be construed as limiting the scope thereof. Indeed, it is contemplated that these teachings will find use in further optimizing the process systems described herein.

In the experimental disclosure which follows, the following abbreviations apply: wt % (weight percent); ° C. (degrees Centigrade); rpm (revolutions per minute); H₂O (water); dH₂O (deionized water); (HCl (hydrochloric acid); aa (amino acid); bp (base pair); kb (kilobase pair); kD (kilodaltons); gm (grams); μg (micrograms); mg (milligrams); ng (nanograms); μl (microliters); ml and mL (milliliters); mm (millimeters); nm (nanometers); μm (micrometer); M (molar); mM (millimolar); μM (micromolar); U (units); V (volts); MW (molecular weight); psi (pounds per square inch); sec (seconds); min(s) (minute/minutes); hr(s) (hour/hours); Q.S. and q.s. (quantity sufficient); OD (optical density); OD₂₈₀ (optical density at 280 nm); OD₆₀₀ (optical density at 600 nm); PAGE (polyacrylamide gel electrophoresis); DO (dissolved oxygen); Di (deionized); phthalate buffer (sodium phthalate in water, 20 mM, pH 5.0); PBS (phosphate buffered saline [150 mM NaCl, 10 mM sodium phosphate buffer, pH 7.2]); Cerestar granular starch (Cargill Foods PFP2200 granular starch; ); Cerestar (Cerestar, Inc., a Cargill Inc., company, Minneapolis, Minn.); AVICELL®(FMC Corporation); SDS (sodium dodecyl sulfate); Tris (tris(hydroxymethyl)aminomethane); w/v (weight to volume); v/v (volume to volume); sipm (standardized liters per minute); ATCC (American Type Culture Collection, Rockville, Md.); Difco (Difco Laboratories, Detroit, Mich.); GIBCO BRL or Gibco BRL (Life Technologies, Inc., Gaithersburg, Md.); Genencor (Genencor International, Inc., Palo Alto, Calif.); Shin Nihon (Shin Nihon, Japan).

In the following examples, additional various media and buffers known to those in the art were used, including the following:

Lactobacilli MRS Media (for inoculum): Difco (Ref#288130):

0.5× Modified Lactobacilli MRS Media w/o Glucose+8% Granular Starch Recipe:

| | | |
|---|---|---|
| Yeast extract (Difco) | 15.0 | g/L |
| Granular starch (Cerestar) | 80.0 | g/L |
| MgSO₄ * 7H₂O | 0.3 | g/L |
| KH₂PO₄ | 0.5 | g/L |
| K₂HPO₄ | 0.5 | g/L |
| Sodium acetate | 0.5 | g/L |
| FeSO₄ * 7H₂O | 0.03 | g/L |
| MnSO₄ * 1H₂O | 0.03 | g/L |
| Mazu DF204 (antifoam) | 1 | ml |
| 1000 × Tiger trace metal | 0.2 | mls stock |
| | | solution |

TM2 Recipe (Per Liter Fermentation Medium):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 5 g, 1000× Modified Tiger Trace Metal Solution 1 ml. All of the components are added together and dissolved in diH₂O. The pH is adjusted to 6.8 with potassium hydroxide (KOH) and q.s. to volume. The final product is filter sterilized with 0.22 u (micron) filter (only do not autoclave).

Murphy III Medium (g/l)

KH₂PO₄ (12 g), K₂HPO₄ (4 g), MgSO₄.7H₂O (2 g), DIFCO Soytone (2 g) citrate (0.1 g), fructose (5 g), (NH₄)₂SO₄ (1 g), nicotinic acid (0.02 g), 0.4 g/l FeCl₃.6H2O (5 ml), and Pho salts (5 ml).

1000× Modified Tiger Trace Metal Solution:

Citric Acids*H₂O 40 g, MnSO₄*H₂O 30 g, NaCl 10 g, FeSO₄*7H₂O 1 g, CoCl₂*6H₂O 1 g, ZnSO*7H₂O 1 g, CuSO₄*5H₂O 100 mg, H₃BO₃ 100 mg, NaMoO₄*2H₂O 100 mg. Each component is dissolved one at a time in Di H₂O, pH to 3.0 with HCl/NaOH, then q.s. to volume and filter sterilize with 0.22 micron filter.

### EXAMPLE 1

### Conversion of Glucose to Gluconate

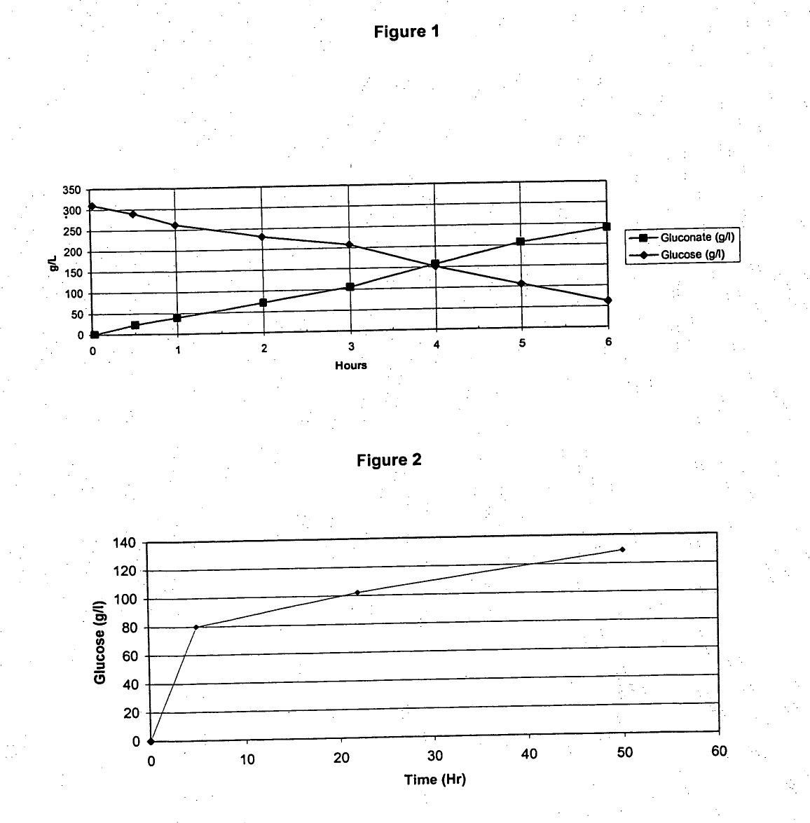

In this Example, experiments conducted to convert glucose to gluconate are describe. First, a 30 wt % glucose solution was produced (115 g of glucose in 275 ml of 50 mM phthalate pH 5.12 in deionized H₂O). This solution was held at 35° C. and 0.3 bar of back-pressure. Then, 2700 U of glucose oxidase and 270 Units of catalase were mixed into the solution at 1100 rpm and 120% DO (under normal temperature and pressure, NTP or ATP) dissolved oxygen in water (“DO”). Upon mixing the enzyme, the DO dipped below 15% of saturation in the reaction medium under operating conditions indicating that with use of 30% glucose, oxygen can be a rate-limiting substrate. Indeed, it appeared that the that enzymes were partially inhibited when tested in solutions that were less than 30% sugar and picked up converting glucose as it went below 20% concentration. Thus, use of 60% sugar solution (i.e., one of the most common sugar feeds utilized in the art) results in inhibition, as well as oxygen transfer challenges. The so results of these experiments are shown in FIG. 1.

### EXAMPLE 2

### Conversion of Starch to Glucose

In this Example, experiments conducted to convert starch to glucose are described. First, a 30% corn starch slurry was made (100 grams of starch [Cerestar] were mixed in 270 ml of 50 mM phthalate buffer, pH 5.0), and was kept at 45° C. Then, the mixture was mixed at 1100 rpm and 150% DO. Then, 250 mg of RSH enzyme (CUCONCTM; Japan; 187 glucoamylase Units/g of powder) were mixed into the solution. This combination resulted in an initial 16 g/l/hr conversion of starch to glucose at pH 5.0 and 45° C. These results indicate that RSH glucoamylase enzyme has excellent kinetics for starch to sugar conversion (See, FIG. 2). However, it is contemplated that lower dosages of RSH glucoamylase will find use in the methods of the present invention to convert starch to glucose. Indeed, in some embodiments in which the 2 g/l/hr production commonly practiced in the art are used, 100 mg of RSH glucoamylase powder (activity/units) per liter of 30% starch stock solution is a sufficient amount to efficiently convert starch to glucose.

In additional experiments to assess the conversion of granular starch to glucose, an experiment was carried out in 1 L orange cap bottle to monitor glucose formation from granular starch using enzymes with glucoamylase activity at desired fermentation pH 6.7 and temperature 34° C.

For this experiment, granular starch in slurry form, for maximum final concentration of 80 g/L glucose, was added to the 1 L bottle (e.g., a 300 mL slurry with 16% glucose equivalent starch was combined with 300 mL of TM2 medium; total of 48 g Cerestar granular starch was added to the 600 ml slurry). The pH of the slurry/broth was adjusted to 6.7 with NH₄OH. The mixture was held at 34° C. for 30 min for germination of any contaminant present in the starch slurry, and then pasteurized at 65° C. for 14 hr. Then, the test enzymes (30 ml UltraFilter concentrate of fermenter supernatant of a Humicola grisea run showing starch hydrolysis activity (i.e., RSH activity) and 0.4 ml of SPEZYME® FRED alpha amylase liquid concentrate (Genencor), as well as 30 mg spectinomycin and 1 mg vitamin B₁₂ (spectinomycin and B₁₂ were added as 0.2 micron filtered solution in DI water). During the reaction, samples were taken from the vessel, centrifuged, and the supernatants refrigerated to terminate the enzyme action. The supernatants were then subjected to HPLC analysis. This experiment monitored saccharification of granular starch by measuring glucose formation. The results indicated that 32.09g/L glucose accumulated in 3 hours. Thus, the conversion of graenular starch to glucose at 10 g/L-hour rate was good for Simultaneous Saccharification and Fermentation (SSF) of granular starch to 1,3-propanediol at 34° C. and pH 6.7.

### EXAMPLE 3

### Conversion of Starch to Gluconate

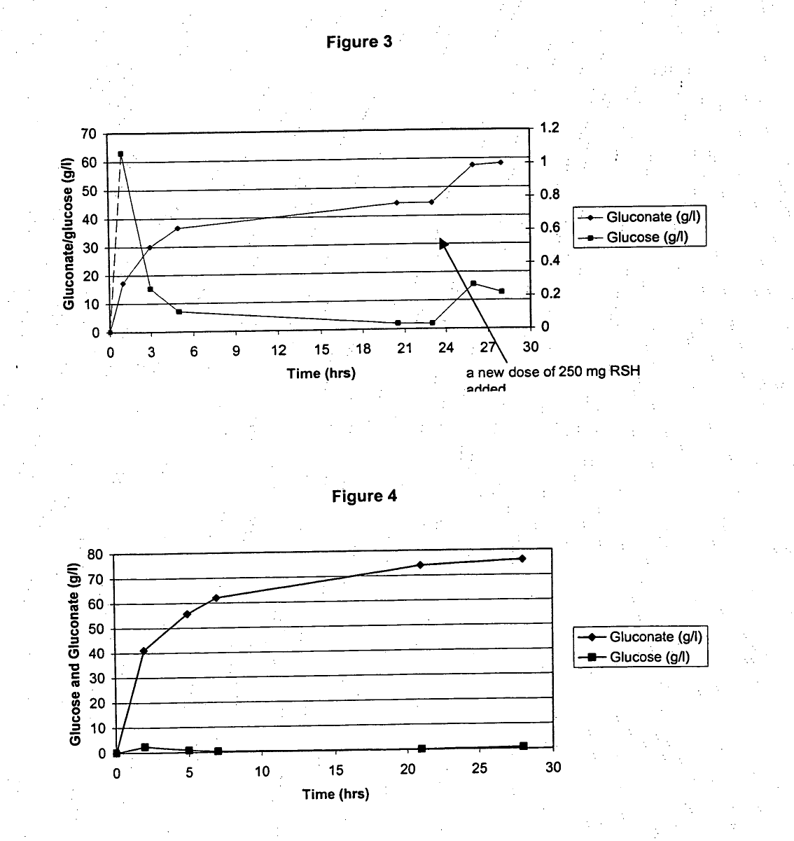

In this Example, experiments conducted to convert starch to gluconate are described. First, a 30% corn starch slurry was made (100 gram of starch in 270 ml of 50 mM phthalate buffer, pH 5.1), and kept at 40° C. Then, under conditions of 1100 rpm and 130 DO, 250 mg of RSH enzyme (CUCONC™; Japan; 187 glucoamylase Units/g of powder), 880 ul of OXYGO® (glucose oxidase; Genencor) and 880 ul of FERMCOLASE® (catalase; Genencor) (1500 U/ml and 1000 U/ml) were mixed into the solution. This resulted in an initial 17 g/l/hr conversion of starch to glucose at pH 5.1-5.2 and 40° C. This result indicates that RSH glucoamylase enzyme has excellent kinetics for starch to sugar conversion under these bioconversion conditions in a bioreactor (See, FIG. 3).

However, in additional embodiments, optimization of conditions helps maximize the long term stability of the system. Additional enzymes needed to convert glucose to gluconate were also determined to work well in unison with this system over the time course used in these experiments, as no glucose accumulation occurred. Thus, these results indicate that the dosage of the RSH enzyme required to run the process at volumetric productivity of 10 g/l/hr is much lower than is required in currently used methods.

### EXAMPLE 4

### Conversion of Starch to Gluconate with Add d DISTILLASE®

In this Example, experiments conducted to convert starch to gluconate using DISTALLASE® in the enzyme mixture are described. First, a 30% corn starch (Cerestar) slurry was prepared in 10 mM acetate buffer (10 mM sodium acetate in water) pH 5.0, and brought to 40° C. Then, under conditions of 1100 rpm and 118 DO, 250 mg of CU CONC™ RSH glucoamylase, 150 ul of DISTILLASE®-L-400 (350 GAU/g; sp 1.15), 1250 ul of OXYGO®, and 1500ul of FERMCOLASE® were added to the solution. This resulted in an initial gluconate production rate of 25 g/l/hr. Thus, it is clear that addition of the DISTILLASE® L-400 glucoamylase enzyme to the reaction mixture helped improve not only the initial rate of gluconate production but also led overall improved conversion of raw corn starch to gluconic acid, as indicated in FIG. 4.

### EXAMPLE 5

### Conversion of Maltodextrin to Glucose

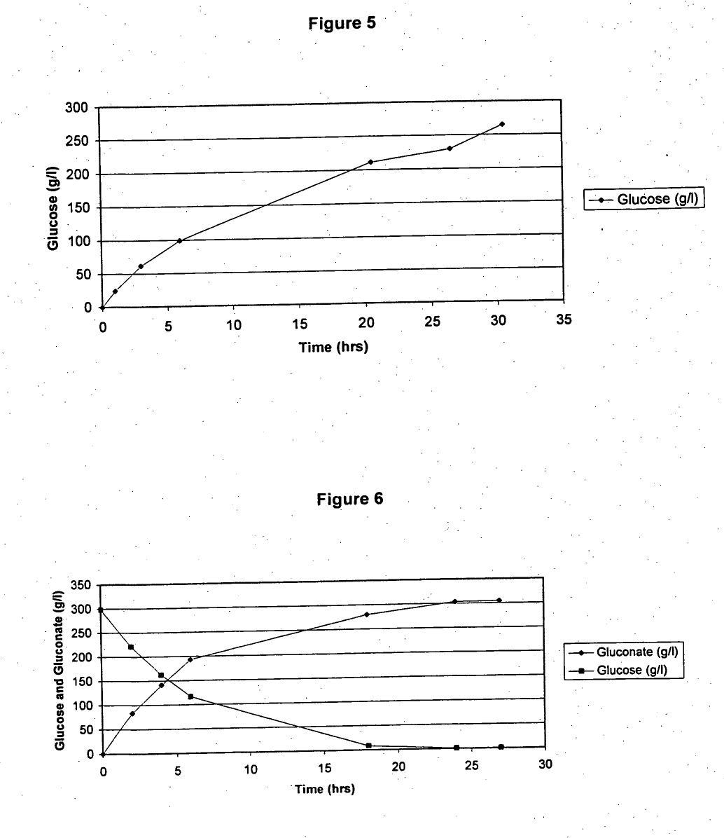

In order to further demonstrate the utility of the methods of the present invention, an alternate substrate was utilized. This substrate, maltodextrin, is also a key sugar source. As shown in FIG. 5, quantitative conversion of maltodextrin to glucose was feasible using OXYGO® and FERMCOLASE® enzymes.

### EXAMPLE 6

### Conversion of Maltodextrin to Gluconate

In addition, the conversion of maltodextrin to gluconate was attempted using low enzyme dosage conditions. In particular, a lower dose of catalase was tested. The results revealed that maltodextrin can be converted to gluconate in a single pot reaction using three enzymes (data not shown). In addition, it was determined that the OPTIMAX® (alpha amylase and pullulanase blend; Genencor) enzyme preparation is less sensitive to hydrogen peroxide, in comparison with CU CONC™ RSH glucoamylase tested in other Examples described herein.

### EXAMPLE 7

### Ratio of OXYGO® and FERMCOLASE® Enzymes

In further experiments, it was determined that a minimal 1:1 ratio of activity basis is desired for maximal productivity and stability of OXYGO® enzyme. As indicated in FIG. 6, complete conversion of glucose to gluconate was demonstrated under these conditions.

### EXAMPLE 8

### Maltodextrin to Gluconate Conversion Using Reestablished Enzyme Dosage

In this experiment, production of gluconate from maltodextrin was achieved to a yield of >50%, at a rate of 7 g/l/hr. Initial conversion rates approached to more than 25 g/l/hr. The dosage level used in this example was 1000 Units of OXYGO® enzyme and FERMCOLASE® enzyme with 200 Units of OPTIMAX® enzyme. This example illustrates the need to utilize the correct enzyme types to achieve the bioconversion.

### EXAMPLE 9

### Optimization of Enzyme Dosages to Improve the Overall Conversion Efficiency

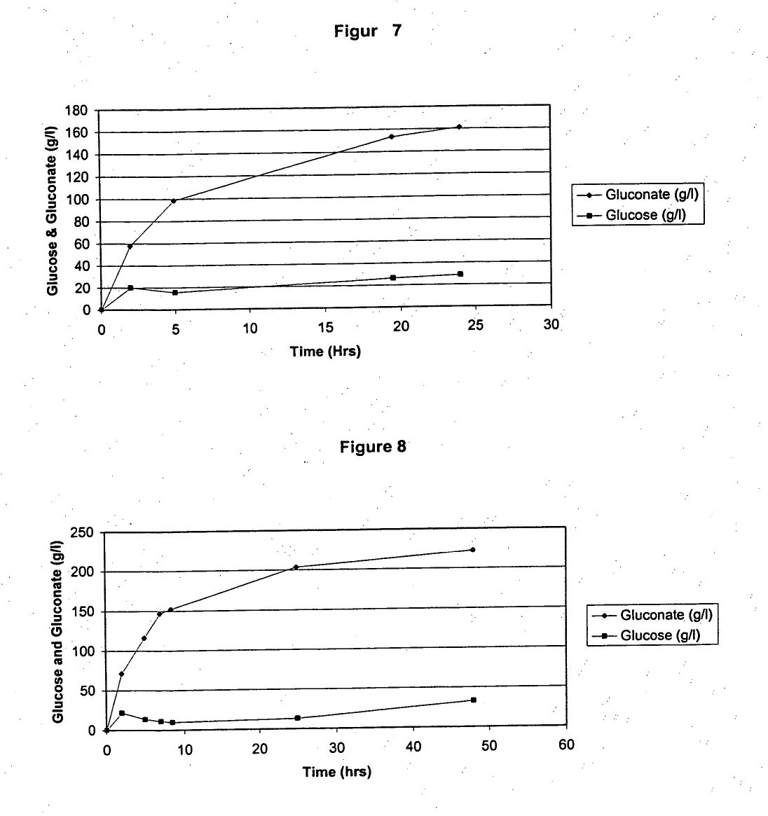

In these experiments, the production yield and volumetric productivity of gluconate from maltodextrin reached to over 80% and 8 g/l/hr by further optimizing the dosage of OPTIMAX® enzyme (See, FIG. 8). The dosage level used in this example was 1250 Units of OXYGO® and 1000 Units FERMCOLASE® with 200 Units of OPTIMAX®. This example illustrates the need to include the correct enzyme type(s) and the dosage level optimization to achieve desired bioconversion.

### EXAMPLE 10

### Comparison Between Raw Corn Starch and Raw Wheat Starch

In order to further demonstrate the utility of the methods of the present invention, an alternate starch source was examined. This substrate, raw wheat starch, is also a key sugar source. As shown in FIG. 9, wheat starch can also be efficiently converted to gluconate using OXYGO®, FERMCOLASE®, DISTILLASE®, and CU CONC RSH glucoamylase enzymes. Indeed, the results indicate that wheat starch is more amenable to bioconversion than corn starch when compared for the similar bioconversion time.

### EXAMPLE 11

### Conversion of Starch to Lactic Acid

This experiment was carried out in 1 L bioreactor to monitor lactate formation from granular starch using enzymes with glucoamylase activity at desired fermentation pH 6.4 and temperature 34° C. In this experiment, granular starch in slurry form (maximum final concentration of 80 g/L glucose) in the 0.5× modified Lactobacilli medium fermentation medium, was pasteurized (i.e., the mixture was held at 34° C. for 30 min for germination of any contaminant present in the starch slurry, and then pasteurized at 65° C. for 14 hr). This was added to the pre-sterilized 1 L bioreactor. The pH of the slurry/broth was adjusted to 6.4 and controlled at 6.4 with 28% NH₄OH. Then, the desired enzymes (0.4 g of sumizyme CU CONC™; Shin Nihon) were added as 0.2 micron filtered solution (20 ml) in DI water. Then, an inoculum of lactate-producing strain Lactobacillus casei (ATCC 393), taken from a frozen vial, was prepared in Lactobacillus MRS medium (Difco). After the inoculum grew to OD 2.4, measured at 550 nm, in a 1L bioreactor at 34° C. with a nitrogen sparge at 0.6 slpm (standardized liters per minute) flow rate), the contents of the reactor (600 ml) were centrifuged and re-suspended in 45 ml supernatant to transfer the cell pellet (42 ml of OD22 material) as the inoculum for the fermentative bioconversion in a bioreactor. For the duration of the fermentative bioconversion run, nitrogen was sparged at 0.6 slpm, the back pressure was held at 5 psi, the temperature was held at 34° C., pH held at 6.4 by base titration of 28% NH₄OH.

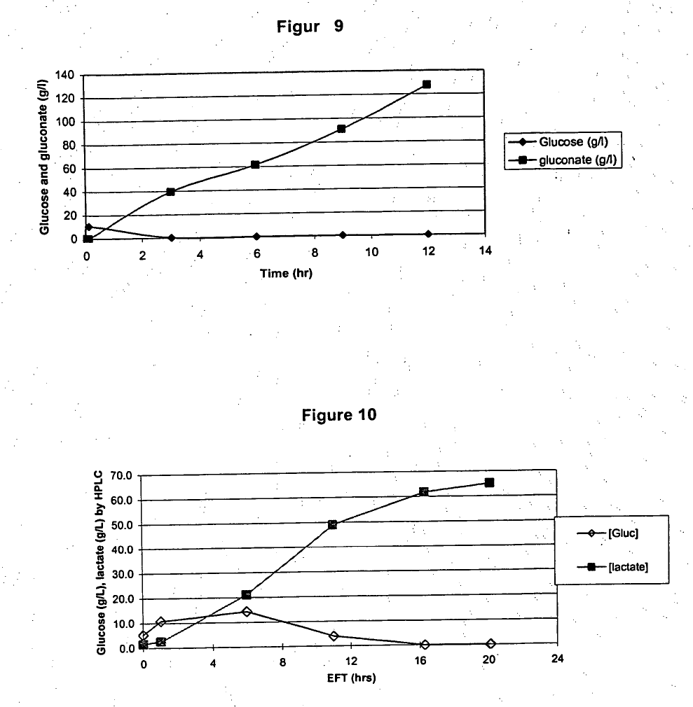

During the reaction, samples were taken from the vessel, centrifuged, and the supernatants were refrigerated to terminate the enzyme action. The supernatant was then subjected to HPLC analysis. This experiment monitored bioconversion of granular starch by measuring glucose formation and its conversion to lactate. In 16.3 hours, accumulation of lactate amounted to 61.75 g/L (FIG. 10).

In addition, the bioconversion of granular starch to lactate was demonstrated to be at a level of 3.79 g/L-hour rate, at a temperature of 34° C., and at pH 6.4.

### EXAMPLE 12

### Conversion of Starch to Succinic Acid

This experiment was carried out in 1 L bioreactor to monitor succinate formation from granular starch using enzymes with glucoamylase activity at desired fermentation conditions of pH 6.7 and temperature 34° C.

For this experiment, raw granular starch in slurry form (maximum final concentration 80 g/L glucose) in 0.5×TM2 fermentation medium, was pasteurized (i.e. the mixture was held at 34° C. for 30 min for germination of any contaminant present in the starch slurry, and then pasteurized at 65° C. for 14 hr). This was added to the pre-sterilized 1 L bioreactor. The pH of the slurry/broth was adjusted to 6.7 and controlled at 6.65 with NH₄OH. Then, the desired enzymes (0.6 g of sumizyme CU CONC; Shin Nihon) were added as 0.2 micron filtered solution (20 ml) in DI water. An inoculum of succinate-producing strain 36 1.6 ppc E. coli, taken from frozen vial, was prepared in TM2+10 g/L glucose medium. After the inoculum grew to OD 0.6, measured at 550 nm, one 600 ml flask was centrifuged and re-suspended in 80 ml supernatant to transfer the cell pellet (80 ml of OD 14.3 material) to the bioreactor. At 3.7 hours in to the run, the air being sparged at 0.6 slpm was switched to nitrogen, which was also sparged at 0.6 slpm.

During the reaction, samples were taken from the vessel, centrifuged and the supernatants were refrigerated to terminate the enzyme action. The supernatant were subjected to HPLC analysis. This experiment monitored bioconversion of granular starch by measuring glucose formation and its conversion to succinate. In 43 hours, accumulation of succinate amounted to 1.46 g/L (FIG. 11). The conversion of granular starch to succinate at 0.034 g/L-hour rate was demonstrated for fermentative bioconversion of granular starch to succinate at 34° C. and pH 6.7.

### EXAMPLE 13

### Conversion of Starch to 1,3-Propanediol

This experiment was carried out in 1L bioreactor to monitor 1,3-propanediol formation from granular starch using enzymes with glucoamylase activity at the desired fermentation pH 6.7 and temperature 34° C.

For this experiment, granular starch in slurry form (for maximum final concentration 80 g/L glucose) in 0.5×TM2 fermentation medium, was pasteurized as described above (i.e., the mixture was held at 34° C. for 30 min for germination of any contaminant present in the starch slurry, and then pasteurized at 65° C. for 14 hr). This was added to the pre-sterilized 1L bioreactor. The pH of the slurry/broth was adjusted to 6.7 and controlled at 6.65 with NH₄OH. Then, the desired enzymes (30 ml UltraFilter concentrate of fermenter supernatant of a Humicola grisea run with starch hydrolysis activity [i.e., RSH glucoamylase activity] and 0.4 ml of SPEZYME® FRED liquid concentrate [Genencor] having alpha amylase activity), and requirements specific for 1,3-propanediol production (30 mg spectinomycin and 2 mg vitamin B₁₂) were added as 0.2 micron filtered solution in DI water. An inoculum of 1,3-propanediol-producing E. coli strain TTaldABml/p109F1 taken from a frozen vial, was prepared in soytone-yeast extract-glucose medium. After the inoculum grew to OD 0.6, measured at 550 nm, two 600 ml flasks were centrifuged and the contents resuspended in 70 ml supernatant to transfer the cell pellet (70 ml of OD3.1 material) to the bioreactor.

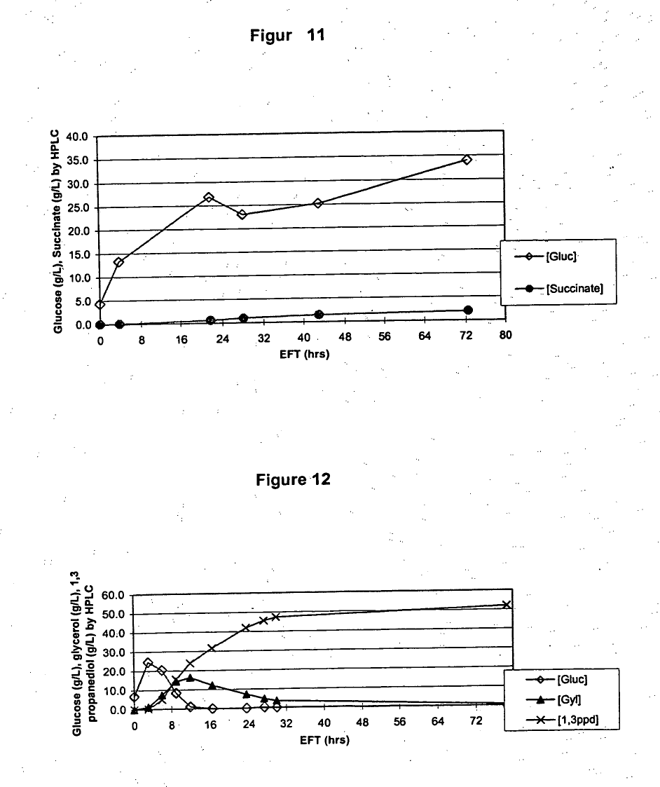

During the reaction, samples were taken from the vessel, centrifuged, and supernatants refrigerated to terminate the enzyme action. The supernatants were then subjected to HPLC analysis. This experiment monitored fermentative bioconversion of granular starch by measuring glucose formation and its conversion to glycerol (1,3-propanediol pathway intermediate) and then to 1,3-propanediol. In 23.5 hours, accumulation of glycerol and 1,3-propanediol amounted to 7.27 and 41.93 g/L, respectively (FIG. 12).

Conversion of granular starch to glycerol and 1,3-propanediol at 1.75 g/L-hour rate was demonstrated for fermentative bioconversion of granular starch to 1,3-propanediol at 34° C. and pH 6.7.

In additional similar experiments, the fermentative bioconversion of granular starch to glycerol was determined at 34° and pH 6.7. In these experiments, glucose formation and its conversion to glycerol were determined. In nine hours, the accumulation of glycerol was found to be 14.93 g/L. The conversion rate of granular starch to glycerol was 1.60 g/L-hour, a good rate for fermentative bioconversion of granular starch. Likewise, the 1.75 g/L-hour rate indicated above, was found to be a good rate for fermentative bioconversion of granular starch to 1,3-propanediol.

### EXAMPLE 14

### Fermentative Bioconversion of Starch to 1,3-Propanediol By CU CONC RSH Glucoamylase

The first experiment was carried out in 1L orange cap bottles to monitor glucose formation from granular starch using enzymes with glucoamylase activity at desired fermentation pH 6.7 and temperature 34° C.

For this experiment, granular starch in slurry form (for maximum final concentration 40 g/L glucose), was added to the 1L bottle (e.g., 300 mL slurry with 8% glucose equivalent starch) and combined with 300 mL of TM2 medium. The pH of the slurry/broth was adjusted to 6.7 with NH₄OH. The mixture was held at 34-35° C. for 30 min for germination of any contaminants present in the starch slurry, and then pasteurized at 65° C. for 14 hr. Then, the desired enzymes (0.6 g Sumizyme CU; Shin Nihon), and requirements specific for 1,3-propanediol production (30 mg spectinomycin and 1 mg vitamin B₁₂) were added as 0.2 micron filtered solution in DI water. During the reaction, samples were taken from the vessel, centrifuged, and the supernatants refrigerated to terminate the enzyme action. The supernatants were then subjected to HPLC analysis. This experiment monitored bioconversion of granular starch by measuring glucose formation. In this experiment, 12.86 g/L glucose accumulated in 6 hours. Conversion of granular starch to glucose at 2 g/L-hour rate was demonstrated for bioconversion of granular starch to 1,3-propanediol at 35° C. and pH 6.7 (data not shown).

In a second experiment, a 1L bioreactor was used to monitor 1,3-propanediol formation from granular starch using enzymes with RSH glucoamylase activity at a desired fermentation pH 6.7 and temperature 34° C. For this experiment, granular starch in slurry form (for maximum final concentration 40 g/L glucose) in TM2 fermentation medium, was sterilized and pasteurized as described above. This mixture was added to the pre-sterilized 1L bioreactor. The pH of the slurry/broth was adjusted to 6.7 and controlled at 6.65 with NH₄OH. The mixture was held at 34° C. for 30 min for germination of any contaminants present in the starch slurry, and then pasteurized at 65° C. for 14 hr. Then, the desired enzyme (0.6 g Sumizyme CU; Shin Nihon), and requirements specific for 1,3-propanediol production (30 mg spectinomycin and 1 mg vitamin B12) were added as 0.2 micron filtered solution in DI water. An inoculum of 1,3-propanediol-producing E. coli strain FMP′ml(1.5 gap)/pSYCO106 taken from a frozen vial, was prepared in soytone-yeast extract-glucose medium. After the inoculum grew to OD 1.1, measured at 550 nm, cells were centrifuged to transfer the cell pellet to the bioreactor.

During the reaction, samples were taken from the vessel, centrifuged, and the supernatants refrigerated to terminate the enzyme action. The supernatants were then subjected to HPLC analysis. This experiment monitored fermentative bioconversion of granular starch by measuring glucose formation and its conversion to glycerol (1,3-propanediol pathway intermediate) and then to 1,3-propanediol. In 5 hours, the accumulation of glycerol and 1,3-propanediol amounted to 2.57 and 0.59 g/L, respectively (FIG. 13).

These results indicated good conversion of granular starch to glycerol and 1,3-propanediol at a 0.63 g/L-hour rate for fermentative bioconversion of granular starch to 1,3-propanediol at 34° C. and pH 6.7.

### EXAMPLE 15

### Fermentative Bioconversion of Starch to Glycerol

This experiment was carried out in a 1L bioreactor to monitor 1,3-propanediol formation from granular starch using enzymes with glucoamylase activity at desired fermentation pH 6.7 and temperature 34° C.

For this experiment, granular starch (Cerestar) in slurry form (for maximum final concentration 80 g/L glucose) in 0.5×TM2 fermentation medium, was pasteurized as described above (i.e., the mixture was held at 34° C. for 30 min for germination of any contaminants present in the starch slurry, and then pasteurized at 65° C. for 14 hr). This mixture was then added to the pre-sterilized 1L bioreactor. The pH of the slurry/broth was adjusted to 6.7 and controlled at 6.65 with NH₄OH. Then, the desired enzymes (30 ml UltraFilter concentrate of a fermenter supernatant obtained from a culture of Humicola grisea showing starch hydrolysis activity [i.e., RSH activity] and also 0.4 ml of SPEZYME® FRED liquid concentrate [Genencor] having alpha amylase activity), and 30 mg spectinomycin were added as 0.2 micron filtered solution in DI water. An inoculum of glycerol producing E. coli strain TTaldABml/p109F1, was prepared in soytone-yeast extract-glucose medium (Difco). After the inoculum grew to OD 0.6, measured at 550 nm, two 600 ml flasks were centrifuged and resuspended in 70 ml supernatant to transfer the cell pellet (70 ml of OD3.1 material) to the bioreactor.

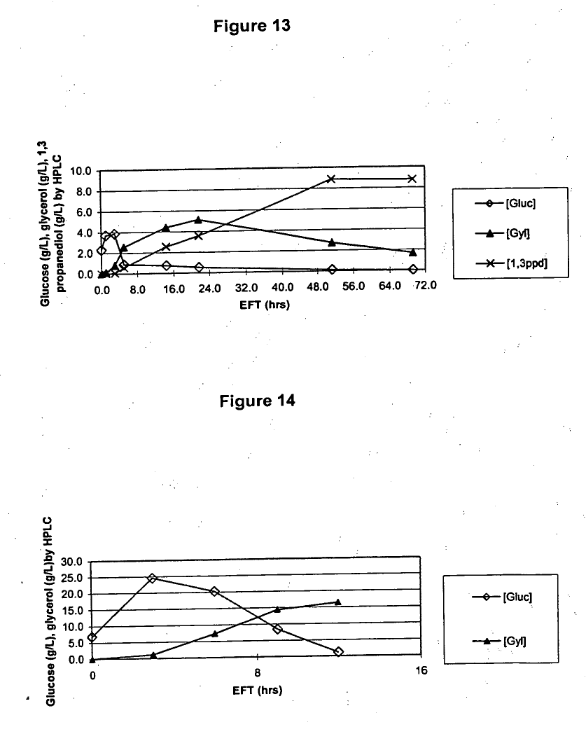

During the reaction, samples were taken from the vessel, centrifuged, and the supernatants refrigerated to terminate the enzyme action. The supernatants were then subjected to HPLC analysis. This experiment monitored fermentative bioconversion of granular starch by measuring glucose formation and its conversion to glycerol. In 9 hours, the accumulation of glycerol amounted to 14.93 g/L (FIG. 14). The conversion of granular starch to glycerol at 1.60 g/L-hour rate was demonstrated for fermentative bioconversion of granular starch to glycerol at 34° C. and pH 6.7.

### EXAMPLE 16

### Conversion of Starch to 2,5-DKG

In this Example, a fermentative bioprocess using corn starch and a RSH glucoamylase is demonstrated to maintain a rate of glucose release which will suffice the maximum production rate of a product such as 2,5-diketo-D-gluconic acid, a precursor molecule of vitamin C, using a microorganism known as Pantoea citrea.

Cerestar raw corn starch and M1Biocon (India) glucoamylase (1786 Gau/g) were used in this study. Pantoea citrea (a Gram-negative bacterial species with periplasmic oxidative dehydrogenases needed for producing oxidative sugar keto acid products such as 2,5-Diketo L-gluconic acid (2,5-DKG) and 2-keto L-gluconic acid 2-KLG from glucose) was used in this Example.

Murphy-III medium was used to grow the cells overnight. A modified Murphy-III medium (see below for formula) was used for the starch to glucose to 2,5-DKG conversion. Shake-flasks and rotary shakers were used in these experiments. Product analyses were performed using HPLC (Water's), and glucose was analyzed enzymatically using the Monarch robotics system (i.e., an instrument known in the art for automated assay work).

Pantoea citrea was inoculated in 100 ml of Murphy-III medium [at 28° C. and 250 rpm overnight. Five-flasks containing 40 ml of deionized water (DI) and 1 gram of raw corn starch (20 g/l final concentration) were pasteurized as described above (i.e. the mixture was held at 34° C. for 30 min for germination of any contaminant present in the starch slurry, and then pasteurized at 65° C. for 14 hr).

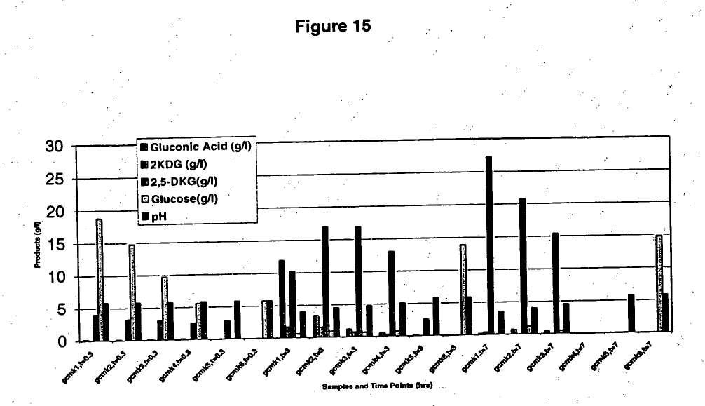

Modified Murphy-III medium was used to provide medium for both further growth of cells and product formation was prepared. Filter-sterilized 10× medium consisted of (per liter), KH₂PO₄, 24 g; K₂HPO₄, 8 g; MgSO₄, 0.16 g; MSG, 1.5 g; (NH₄)₂, 1 g; nicotinic acid; Pho salts (CaCl₂, MnCl₂, NaCl); FeCl₃; pantothenate and tetracycline 20 mg/l. The pH of the medium was adjusted to 5.8 using potassium phosphate. Then, 5 ml of this medium were aseptically added to the shake-flasks containing the starch and water mixture. In another flask, 40 ml of water containing 1 gram of glucose and 5 ml of the modified Murphy-III medium were added aseptically. Then, 5 ml of cell culture which grew to an OD of 21.5 at 550 nm overnight were then added to five-flasks. Flask-1 (GCMK1) thus contained 20 g/l glucose and 5 ml of P. citrea cell culture in modified Murphy-III medium. Flask-2 (GCMK2) contained 1 g of starch, 5mI of cell culture and the reaction was started with addition of 10 units of Biocon glucoamylase. Flask-3 (GCMK3) was the same as flask-2 except it also contained 3 units of glucoamylase. Flask4 (GCMK4) had an added 1 unit of glucoamylase. Flask-5 (GCMK5) was a control, with no glucoamylase added. Flask-6 (GCMK6) was another control, in which 1 unit of glucoamylase was added but no cells were added. At three time points (0.3 hrs, 3 hrs, and 7 hrs) during incubation, 1.5 ml samples were withdrawn from each flask and were centrifuged. The supernatants were then filtered and processed for product analysis, pH, and glucose measurements. The results are shown in FIG. 15.

The results indicated that corn starch is a suitable carbon source in fermentation control and production of 2,5-DKG using P. citrea cells and glucoamylase. Flasks 4 and 6, which contained 1 unit of glucoamylase had similar glucose levels of 5.6 g/l. This glucose level translates to a 20 g/l/hr conversion rate. Thus, Flask-2 with 10 units of glucoamylase had 15 g/l of glucose within 0.3 hr. The results of Flask-1 (with added glucose) were similar to those obtained with Flask-2. The rate of glucose production in Flask-3 correlated well with Flasks 2 and 4. As expected, Flask-5 had no glucose.

At three-hour time point, glucose levels in Flasks 14 dropped below 1 g/l and were converted to oxidative products gluconic acid, 2KDG and 2,5-DKG. It was interesting to note that Flask-2, 3 and 4, with controlled release of glucose, demonstrated greater end-product formation-whereas Flask-1 with excess glucose produced lower levels of end product formation, but still had higher product intermediate concentrations. Control Flasks 5, and 6 behaved as expected.

By the seven hour sampling time point, each of Flasks 1-4 produced the expected product levels. In addition, the pH dropped in Flasks 1-4 and the trends were as expected based on the product (sugar acid) formation

### EXAMPLE 17

### Bioconversion of Cellulosic Biomass to Gluconic Acid

As indicated in this Example, cellulose derived from biomass such as AVICEL® (FMC Corporation) and corn stover can be converted to a desired end-product using biocatalytic systems. This method for converting biomass overcomes product inhibition of cellulolytic enzymes during the conversion of biomass to glucose. This process converts the cellulolytic end-products concomitantly to the desired final product so that inhibition of cellulolytic enzymes is minimized. Cellulosic end-products such as glucose, xylose and cellobiose are produced, but are converted at the same time and rate to the final product, thereby allowing minimal accumulation of these end products which are also inhibitory to cellulolytic enzymes. Thus, the present method provides improved productivity and yield of the desired end-product

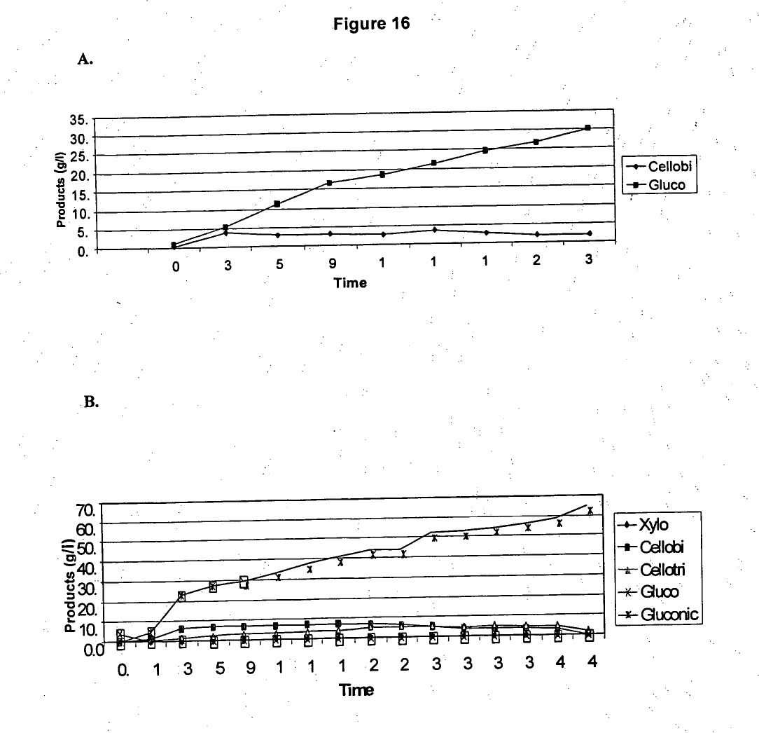

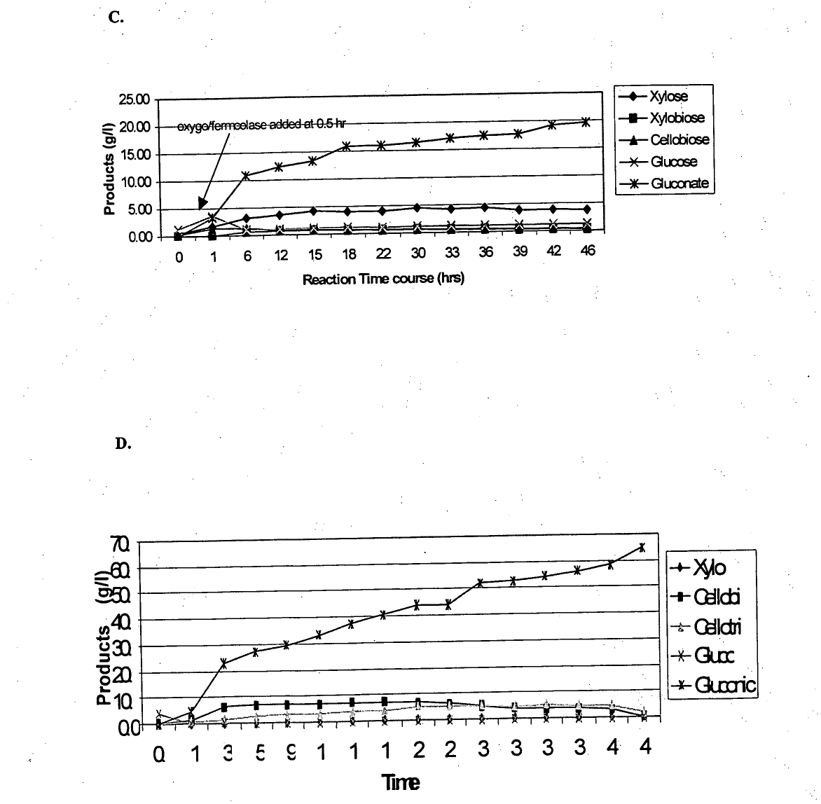

In these experiments, cellulose (AVICEL®; 30 g 10 wt %) and corn stover ( 30 g, 10 wt %) were tested in separate experiments, in 270 g of 50 mM citrate buffer pH 5.0 in a 1 liter bioreactor at 45° C. equipped with pH, stirring, temperature, foam and oxygen control. Conversion of cellulose to glucose was started by adding 10 ml (dosed at 30 mgs of total protein per gram of cellulose) of SPEZYME® CP (Genencor) and the degree of hydrolysis was measured over the course of the reaction. In a subsequent experiment, 1.5 ml OXYGO® glucose oxidase (Genencor) and 2 ml FERMCOLASE® catalase (Genencor) were mixed along with 10 ml SPEZYME® CP (Genencor) were added to the cellulose and corn stover. These enzymes were found to convert the cellulose and corn stover to gluconic acid at an improved rate, as compared to the rate of glucose production from cellulose in a control experiment. This allowed the steady-state concentration of glucose in the reaction to remain at an essentially non-existent level. The gluconic acid concentration was measured using HPLC and the degree of hydrolysis was back calculated. The results established that in the same period of time where 30 g/l glucose was made from AVICEL®, in the control experiment (See, FIGS. 16A and 16B), over 50 g/l gluconic acid from AVICEL® was made using enzyme blend of OXYGO®, FERMCOLASE®, and SPEZYME®. In a 48 hr time frame, 60 wt % tech grade AVICEL® (Lattice 20) was converted to gluconic acid (FIG. 16B). It was observed that by keeping the cellulosic end-product concentration at a minimum, it is possible to keep the cellulose hydrolyzing enzymes stable during the time course of the reaction.

### EXAMPLE 18

### Fermentative Bioconversion of Biomass to 1,3-Propanediol

This Example experiments to determine the suitability of using bioconversion to produce 1,3-propane diol from biomass are described. These experiments were carried out in a 2L tri-baffled Erylenmeyer flask to monitor glucose formation from cellulose (technical grade, AVICEL® Lattice 20) using enzymes with cellulase activity at desired fermentation pH 6.7, at 34° C.

For this experiment, cellulose in slurry form (for maximum final concentration 100 g/L glucose), was added to the 2L flask (e.g., 200 mL slurry with 20% cellulose) was combined with 200 mL of TM2 medium (to give a 100 g/L glucose equivalent). The pH of the slurry/broth was adjusted to 6.7 with NH₄OH. The mixture was sterilized at 121° C. for 30 min. Then, the desired enzyme (13 ml SPEZYME® CP; Genencor), and requirements specific for 1,3-propanediol production (20 mg spectinomycin and 1 mg vitamin B12) were added as a 0.2 micron filtered solution in DI water. During the reaction, samples were taken from the vessel, centrifuged, and the supernatants refrigerated to terminate enzyme action. The supernatants were subjected to HPLC analysis. This experiment monitored degradation of biomass (cellulose) by measuring glucose formation. It was determined that 12.19 g/L glucose accumulated in 98.7 hours. Conversion of biomass to glucose at a 0.12 g/L-hour rate was demonstrated for bioconversion of biomass to 1,3-propanediol at 34° C. and pH 6.7 (data not shown).

Subsequently, an experiment was carried out in a 1L bioreactor to monitor glucose formation from cellulose (technical grade, AVICEL®) using enzymes with cellulase activity at desired fermentation pH 6.7 and temperature 34° C. In this experiment, biomass (cellulose) in slurry form (for maximum final concentration 100 g/L glucose) in TM2 fermentation medium, was sterilized in the 1L bioreactor. The pH of the slurry/broth was adjusted to 6.7 and controlled at 6.65 with NH₄OH. The mixture was sterilized at 121° C. for 30 mins. Then, the desired enzymes (22 ml SPEZYME® CP; Genencor), and requirements specific for 1,3-propanediol production (30 mg spectinomycin and 1 mg vitamin B12) were added as 0.2 micron filtered solution in DI water. An inoculum of 1,3-propanediol-producing E. coli strain TTaldABml/p109f1 WS#2 taken from a frozen vial, was prepared in soytone-yeast extract-glucose medium (Difco). After the inoculum grew to OD 1.2, measured at 550 nm, 60 mls of broth were transferred to the bioreactor.

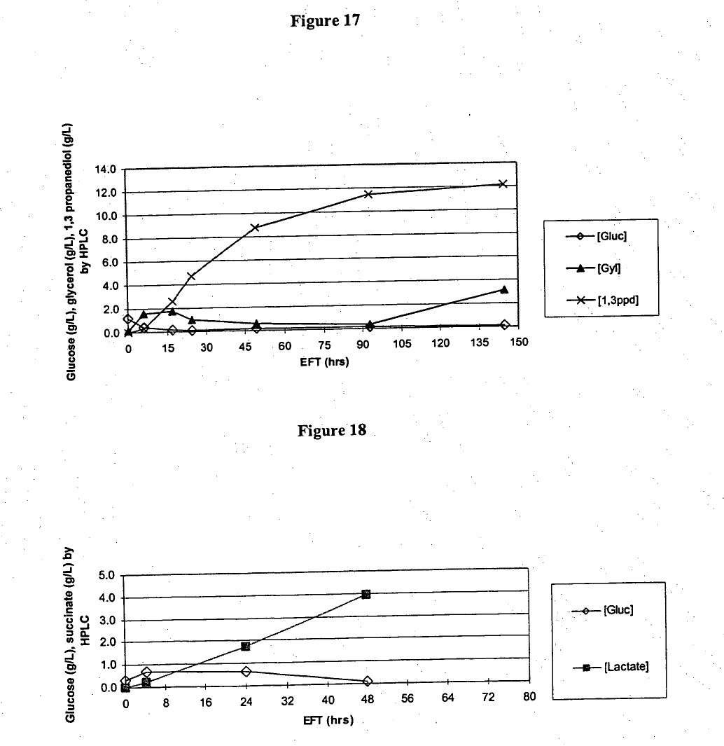

During the reaction, samples were taken from the vessel, centrifuged, and the supernatants refrigerated to terminate the enzyme action. The supernatants were subjected to HPLC analysis. This experiment monitored fermentative bioconversion of biomass to 1,3-propanediol by measuring glucose formation and its conversion to glycerol (1,3-propanediol pathway intermediate) and then to 1,3-propanediol. In 24.4 hours, the accumulation of glycerol and 1,3-propanediol amounted to 1.02 and 4.73 g/L, respectively (See, FIG. 17).

The conversion of biomass to glycerol and 1,3-propanediol at 0.24 g/L-hour rate was demonstrated for fermentative bioconversion of biomass to 1,3-propanediol at 34° C. and pH 6.7.

### EXAMPLE 19

### Fermentative Bioconversion of Biomass to Lactic Acid

This experiment was carried out in a 1L bioreactor to monitor glucose formation from cellulose (technical grade, AVICEL® Lattice 20) using an enzyme with cellulase activity at desired fermentation pH 6.4 and temperature 34° C., and the subsequent conversion to lactate using the lactate producing strain Lactobacillus casei.

For this experiment, biomass (cellulose) in slurry form (for maximum final concentration 100 g/L glucose) in the modified Lactobacilli MRS medium, was sterilized in the 1L bioreactor. The pH of the slurry/broth was adjusted to 6.4 and controlled at 6.4 with 28% NH₄OH. The mixture was sterilized at 121° C. for 30 min. After cooling to a run temp of 34° C., the desired enzyme (22 ml SPEZYME® CP; Genencor) was added as 0.2 micron filtered solution in DI water. An inoculum of lactate producing strain Lactobacillus casei (ATCC 393), taken from a frozen vial, was prepared in Lactobacilli MRS medium (Difco). After the inoculum grew to OD 2.7, measured at 550 nm, in a 1L bioreactor at 34° C. with a nitrogen sparge at 0.6 slpm, the contents of the reactor (600 ml) were centrifuged and re-suspended in 50mI supernatant to transfer the cell pellet (46 ml of OD 24.2 material) as the inoculum for the SDC bioreactor.

During the reaction, samples were taken from the vessel, centrifuged, and the supernatants were refrigerated to terminate the enzyme action.. The supernatants were subjected to HPLC analysis. This experiment monitored fermentative bioconversion of biomass to lactate by measuring glucose formation and its conversion to lactate. In 48 hours, accumulation of lactate amounted to 3.93 g/L (FIG. 18).

### EXAMPLE 20

### Fermentative Bioconversion of Biomass to Succinic Acid

This experiment was carried out in a 1L bioreactor to monitor glucose formation from cellulose (technical grade, AVICEL® Lattice 20) using enzymes with cellulase activity at desired fermentation pH 6.7 and temperature 34° C., and the subsequent conversion to succinate, using the succinate producing strain, 36 1.6 ppc (E. coli).

For this experiment, biomass (cellulose) in slurry form (for maximum final concentration 100 g/L glucose) in the TM2 fermentation medium, was sterilized in the 1L bioreactor. The pH of the slurry/broth was adjusted to 6.7 and controlled at 6.65 with NH₄OH. The mixture was sterilized at 121° C. for 30 min. After cooling to a run temp of 34° C., the desired enzyme (22 ml SPEZYME® CP; Genencor) were added as 0.2 micron filtered solution in DI water. An inoculum of succinate-producing strain 36 1.6ppc E. coli, taken from a frozen vial, was prepared in TM2+10 g/L glucose medium. After the inoculum grew to OD 0.85, measured at 550 nm, the contents of one 600 ml flask was centrifuged and re-suspended in 60 ml supernatant to transfer the cell pellet (60 ml of OD 9.3 material) to the bioreactor. For the duration of the run, nitrogen was sparged at 0.6 slpm

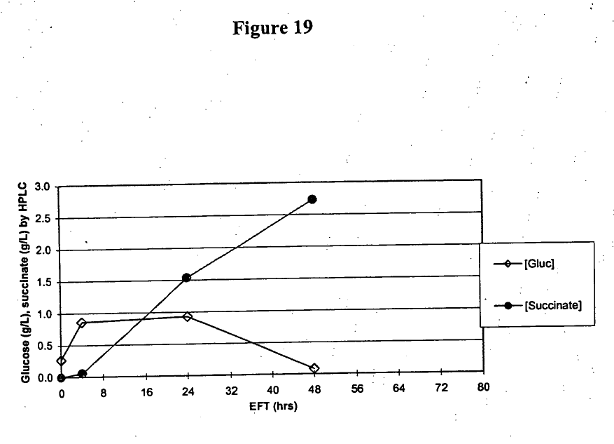

During the reaction, samples were taken from the vessel, centrifuged and supernatants were refrigerated to terminate the enzyme action. The supernatant was subjected to HPLC analysis . This experiment monitored fermentative bioconversion of biomass to succinate by measuring glucose formation and its conversion to succinate (See, FIG. 19). In 48 hours, accumulation of succinate amounted to 2.73 g/L.

1. A method for producing an end-product comprising the steps of:
a) contacting a carbon substrate and at least one substrate-converting enzyme to produce an intermediate; and
b) contacting said intermediate with at least one intermediate-converting enzyme, wherein said intermediate is substantially all converted by said intermediate enzyme to said end-product.

2. The method of claim 1, wherein said intermediate-converting enzyme is a microbial enzyme.

3. The method of claim 2, wherein said intermediate-converting microbial enzyme is secreted by a microorganism in contact with said intermediate.

4. The method of claim 1, wherein said substrate-converting enzyme is a microbial enzyme.

5. The method of claim 4, wherein said substrate-converting microbial enzyme is secreted by a microorganism in contact with said substrate.

6. The method of claim 1, wherein said intermediate-converting enzyme and said substrate-converting enzyme are produced by microorganisms of the same species.

7. The method of claim 1, wherein said intermediate-converting enzyme and said substrate-converting enzyme are produced by microorganisms of the different species.

8. The method of claim 1, wherein concentration level of said intermediate is maintained at a level below that which triggers catabolite repression effects upon the conversion of said intermediate to said end-product.

9. The method of claim 1, wherein concentration level of said intermediate is maintained at a level below that which triggers enzymatic inhibition effects upon the conversion of said intermediate to said end-product.

10. The method of claim 1, wherein said intermediate is converted to said end-product at a rate sufficient to maintain the concentration of said at less than 0.25%.

11. The method of claim 1, wherein said substrate is selected from the group consisting of biomass and starch.

12. The method of claim 1, wherein said intermediate is selected from the group consisting of hexoses and pentoses.

13. The method of claim 12, wherein said hexose is glucose.

14. The method of claim 1 wherein said end-product is selected from the group consisting of 1,3-propanediol, gluconic acid, glycerol, succinic acid, lactic acid, 2,5-diketo-D-gluconic acid, gluconate, glucose, alcohol, and ascorbic acid intermediates.

15. The method of claim 1, wherein said contacting said substrate and substrate-converting enzyme further comprises bioconverting said substrate to produce said intermediate.

16. A method for producing an end-product comprising the steps of:
a) contacting a carbon substrate and at least one substrate-converting enzyme to produce an intermediate; and
b) contacting said intermediate with at least one intermediate-converting enzyme, wherein said intermediate is substantially all converted by said intermediate enzyme to said end-product, and wherein the presence of said end-product does not inhibit the further production of said end-product.

17. The method of claim 16, wherein said intermediate-converting enzyme is a microbial enzyme.

18. The method of claim 16, wherein said intermediate-converting microbial enzyme is secreted by a microorganism in contact with said intermediate.

19. The method of claim 16, wherein said substrate-converting enzyme is a microbial enzyme.

20. The method of claim 16, wherein said substrate-converting microbial enzyme is secreted by a microorganism in contact with said substrate.

21. The method of claim 16, wherein said intermediate-converting enzyme and said substrate-converting enzyme are produced by microorganisms of the same species.

22. The method of claim 16, wherein said intermediate-converting enzyme and said substrate-converting enzyme are produced by microorganisms of the different species.

23. A method for producing an end-product comprising the steps of:
a) contacting a carbon substrate and at least one substrate-converting enzyme to produce an intermediate; and
b) contacting said intermediate with at least one intermediate-converting enzyme, wherein said intermediate is substantially all converted by said intermediate enzyme to said end-product, and wherein the presence of said substrate does not inhibit the further production of said end-product.

24. The method of claim 23, wherein said intermediate-converting enzyme is a microbial enzyme.

25. The method of claim 23, wherein said intermediate-converting microbial enzyme is secreted by a microorganism in contact with said intermediate.

26. The method of claim 23, wherein said substrate-converting enzyme is a microbial enzyme.

27. The method of claim 23, wherein said substrate-converting microbial enzyme is produced is secreted by a microorganism in contact with said substrate.

28. The method of claim 23, wherein said intermediate-converting enzyme and said substrate-converting enzyme are produced by microorganisms of the same species.

29. The method of claim 23, wherein said intermediate-converting enzyme and said substrate-converting enzyme are produced by microorganisms of the different species.
